(19) United States
(12) Patent Application Publication
(10) Pub. No.: US 2023/0235372 A1
(43) Pub. Date: Jul. 27, 2023

(54) AB-INITIO, TEMPLATE-INDEPENDENT SYNTHESIS OF NUCLEIC ACIDS USING THERMOSTABLE ENZYMES

(71) Applicant: SYNHELIX, Evry-Courcouronnes (FR)

(72) Inventors: Irina RANDRIANJATOVO-GBALOU, Evry-Courcouronnes (FR); Ahmed SAID, Evry-Courcouronnes (FR); Renaud RAHIER, Evry-Courcouronnes (FR)

(21) Appl. No.: 18/009,667

(22) PCT Filed: Jun. 11, 2021
(86) PCT No.: PCT/EP2021/065859

§ 371 Date: Dec. 9, 2022

Related U.S. Application Data
(60) Provisional application No. 63/038,168, filed on Jun. 12, 2020.

(30) Foreign Application Priority Data
Aug. 6, 2020 (EP) 20305904.3

(51) Int. Cl. C12P 19/34; C12N 9/12; C12N 15/63

(52) U.S. Cl. CPC C12P 19/34; C12Y 207/07031; C12N 9/1264; C12N 9/1252; C12N 15/63

(57) ABSTRACT

The invention relates to the field of nucleic acid synthesis or sequencing, more specifically to methods for ab-initio synthesis of nucleic acids, comprising contacting a nucleotide with a free 3′-hydroxyl group, with at least one nucleoside triphosphate, or a combination of nucleoside triphosphates, in the presence of an archaeal DNA primase or a functionally active fragment and/or variant thereof, thereby covalently binding said nucleoside triphosphate to the free 3′-hydroxyl group of the nucleotide. It also relates to isolated functionally active fragments of archaeal DNA primases which are capable of both ab-initio single-stranded nucleic acid synthesis activity and template-independent terminal nucleotidyl transferase activity.

### FIELD OF THE INVENTION

The invention relates to the field of nucleic acid synthesis or sequencing, more specifically to methods for ab-initio synthesis of nucleic acids, comprising contacting a nucleotide with a free 3′-hydroxyl group, with at least one nucleoside triphosphate, or a combination of nucleoside triphosphates, in the presence of an archaeal DNA primase or a functionally active fragment and/or variant thereof, thereby covalently binding said nucleoside triphosphate to the free 3′-hydroxyl group of the nucleotide.

It also relates to isolated functionally active fragments of archaeal DNA primases which are capable of both ab-initio single-stranded nucleic acid synthesis activity and template-independent terminal nucleotidyl transferase activity.

### BACKGROUND OF THE INVENTION

Template-independent synthesis of nucleic acids represents a major industrial challenge. Many industries are capable of synthesizing DNA or RNA strands by chemical means; however, they have quickly experienced the limits of these manufacturing processes. Today, the gold method for chemical synthesis of nucleic acids is the phosphoramidite method, developed in the 1980's, and later enhanced with solid-phase supports and automation (Beaucage & Caruthers, 1981. Tetrahedron Lett. 22(20):1859-62; McBride & Caruthers, 1983. Tetrahedron Lett. 24(3):245-8; Beaucage & Iyer, 1992. Tetrahedron. 48(12):2223-2311).

However, this method shows major limitations: first, nucleic acids with no more than around 250 nucleotides can be synthetized. Second, the phosphoramidite method requires the use of organic solvents which can be carcinogens, reproductive hazards, and neurotoxins; while synthetic byproducts can further be toxic and polluting.

In order to overcome these problems, a new method of template-independent, sequence-controlled nucleic acid synthesis by enzymatic means has recently been developed. It is based on the use of enzymes capable of terminal transferase activity, such as X-family DNA polymerases (including terminal deoxynucleotidyl transferases [TdT] or DNA polymerase μ) and A-family DNA polymerases (including DNA polymerase θ) (Kent et al., 2016. Elife. 5:e13740).

However, a common feature of all the enzymes mentioned above is their requirement of a single-stranded DNA primer, of at least 4 nucleotides long, to initiate nucleic acid synthesis. Such a condition implies that an extra synthetic initiator sequence needs to be added to the reaction media and needs to be removed after synthesis. Thus, nucleic acid synthesis using the above-mentioned enzymes engages extra costs and time for initiator sequence synthesis and removal, using chemical, biochemical and/or physical methods.

In addition, to control such processes, modified nucleoside triphosphates bearing a blocking group at their 3′-OH end (called “terminating nucleoside triphosphates”, “3′-blocked nucleoside triphosphates” or “3′-protected nucleoside triphosphates”) are generally used (Tauraité et al., 2017. Molecules. 22(4):672; WO2017216472; WO2018102554). Such nucleoside triphosphates are said “reversible terminating” because an oligonucleotide formed by the addition of such nucleoside triphosphate under enzymatic activity cannot be further extended until the 3′-OH blocking group is removed. In this way, only one nucleotide is temporarily incorporated into the growing nucleic acid strand, even in homopolymeric regions. Among the commercially-available reversible terminating nucleoside triphosphates with a 3′-OH blocking group, the oxime-blocked (3′-ONH₂) nucleoside triphosphates developed by Benner and colleagues (Hutter et al., 2010. Nucleosides Nucleotides Nucleic Acids. 29(11):879-895), the 3′-allyl nucleoside triphosphates produced by Ju and colleagues (Guo et al., 2010. Acc Chem Res. 43(4):551-563) or the 3′-azidomethyl nucleoside triphosphates (Guo et al., 2008. Proc Natl Acad Sci USA. 105(27):9145-9150) are commonly used to control the enzymatic synthesis of oligonucleotides.

Since the emergence of next-generation nucleic acids synthesis, the field of reversible terminating nucleoside triphosphates is also booming. Several patents and studies relate the preparation of theses reversible terminating analogs (WO2018102554; U.S. Pat. No. 8,034,923), and although nucleoside triphosphate purification methods have evolved considerably over time, it is extremely complicated, if not impossible, to obtain 100% of nucleoside triphosphates with a 3′-OH blocking group in the reaction pre-mix.

As a case study, the synthesis of a functional oligonucleotide (e.g., DNA or RNA aptamer, ribozyme or DNAzymes, riboswitches, etc.) requires having the exact nucleotide sequence with the highest fidelity. One uncontrolled addition of a single nucleotide can have a devastating effect on the secondary structure of the functional oligonucleotide and thus alter its biological function. Assuming that x % of the nucleoside triphosphates in the reaction pre-mix lack a 3′-OH blocking group, a bias of x % of the synthesized oligonucleotide incorporating the wrong nucleotide at each cycle would be artificially introduced. This would increase exponentially with the number of cycles to be performed. For instance, the group of Benner has experienced the incorporation of unprotected nucleoside triphosphates, present in an amount of around 3% in the sample (Hutter et al., 2010. Nucleosides Nucleotides Nucleic Acids. 29(11):879-895; Supplementary material S34).

On that basis, the Inventors relate here the importance of developing a method of nucleoside triphosphate clean-up that will allow to obtain terminating nucleoside triphosphate pools with up to 100% of purity.

Several processes are already described in the art, and commonly used to clean-up unprotected nucleoside triphosphates in terminating nucleoside triphosphate pools.

The simplest and classical way is to perform a PCR using a nucleic acid template anchored to a solid support, leading to an easy separation of the PCR product from the terminating nucleoside triphosphates. Indeed, during the PCR, the DNA polymerase will only take in charge those nucleoside triphosphates that have a free 3′-OH end (i.e., unprotected nucleoside triphosphates). Thus, at the end of the reaction, the remaining pool of terminating nucleoside triphosphates (which could not be used during the PCR) will be enriched. Even if this process is effective, it remains considerably expensive since it requires the purchase of a nucleic acid template and primers for each nucleotide to perform the polymerase assay. In addition, the traditional Taq DNA polymerase used for PCR reactions can only incorporate the four natural deoxynucleotides (dATP, dTTP, dGTP, dCTP). Thus, a PCR clean-up will not guarantee the elimination of other nucleoside triphosphates (such as ribonucleotides or artificial nucleoside triphosphates), intermediate analogs (such as acetone-oximes, etc.) or co-products of the reaction.

Another technique relies on the use of terminal transferase-like enzymes (TdT) that are capable of adding nucleotides to a nucleic acid primer without a template strand to copy. Indeed, such an enzyme has the ability to bind to a single stranded DNA and to incorporate several unprotected nucleoside triphosphates. The nucleic acid primer could further be attached to a solid support, to facilitate the purification of the free terminating nucleoside triphosphates. Experiments conducted in our laboratory have shown that commercially-available TdT can add about 400 nucleotides to a single-stranded DNA primer. This enzyme could thus be used to carry out a nucleoside triphosphate clean-up method, but major drawbacks remain, as (1) it is still necessary to add a greater amount of single-stranded DNA primer to exhaust the totality of the unprotected nucleoside triphosphates; and (2) the optimal temperature range by which the TdT is used (37-45° C.) makes it difficult if not ineffective to purify some nucleoside triphosphates, in particular dGTP. Indeed, some G-quadruplex structures can form and might conduct to the early termination of a poly-G nucleic acid synthesis using those unprotected dGTP.

It is therefore necessary to find alternative means and methods for nucleoside triphosphate clean-up overcoming these issues. Several years ago, Forterre and colleagues have characterized the biochemical properties of an archaeal DNA primase named “PolpTN2”, isolated from the Thermococcus nautili (formerly reported as Thermococcus nautilus) plasmid pTN2 (Gill et al., 2014. Nucleic Acids Res. 42(6):3707-3719). The native full-length enzyme has been shown to exhibit some strictly dNTPs-dependent DNA primase, DNA polymerase activities while a truncated version, herein named PolpTN2_{Δ311-923}, has been shown to exhibit a terminal nucleotidyl transferase activity.

In addition, Béguin et al. have demonstrated that a combination of the full length PolpTN2 primase and the PolB DNA polymerase in presence of deoxynucleotide triphosphates leads to the ab-initio synthesis of long double-stranded DNA fragments (i.e., without template DNA nor oligonucleotide primer). However, this phenomenon requires the presence of both enzymes and is not observed when only PolpTN2 is reacted with a dNTP mix (Béguin et al., 2015. Extremophiles. 19(1):69-76).

Here, the Inventors surprisingly show that PolpTN2_{Δ311-923} is capable of ab-initio single-stranded nucleic acid synthesis activity, a surprising activity which was not previously described in any members of the archaeo-eukaryotic primase (AEP) superfamily.

Based on these findings, the present invention offers efficient means and methods for ab-initio synthesis and functionalization of nucleic acids, as well as nucleoside triphosphate clean-up, overcoming the issues set out above.

### SUMMARY

The present invention relates to a method for ab-initio single-stranded nucleic acid synthesis, comprising contacting the free 3′-hydroxyl group of a nucleotide with at least one nucleoside triphosphate, or a combination of nucleoside triphosphates, in the presence of a primase domain of an archaeal DNA primase belonging to the primase-polymerase family or a functionally active variant thereof capable of both ab-initio single-stranded nucleic acid synthesis activity and template-independent terminal nucleotidyl transferase activity, thereby covalently binding said nucleoside triphosphate to the free 3′-hydroxyl group of the nucleotide.

In one embodiment, said archaeal DNA primase or the functionally active variant thereof is from an archaeon of the Thermococcus genus.

In one embodiment, said archaeal DNA primase belonging to the primase-polymerase family or the functionally active variant thereof is selected from the group consisting of Thermococcus nautili sp. 30-1 DNA primase, Thermococcus sp. CIR10 DNA primase, Thermococcus peptonophilus DNA primase, and Thermococcus celericrescens DNA primase.

In one embodiment, said archaeal DNA primase belonging to the primase-polymerase family or the functionally active variant thereof is:
Thermococcus nautili sp. 30-1 DNA primase having the amino acid sequence of SEQ ID NO: 1;
Thermococcus sp. CIR10 DNA primase having the amino acid sequence of SEQ ID NO: 14;
Thermococcus peptonophilus DNA primase having the amino acid sequence of SEQ ID NO: 17; or
Thermococcus celericrescens DNA primase having the amino acid sequence of SEQ ID NO: 19.

In one embodiment, said primase domain of an archaeal DNA primase belonging to the primase-polymerase family is the primase domain of:
the Thermococcus nautili sp. 30-1 DNA primase having the amino acid sequence of any one of SEQ ID NOs: 2 to 13; or
the Thermococcus sp. CIR10 DNA primase having the amino acid sequence of any one of SEQ ID NOs: 15 or 16; or
the Thermococcus peptonophilus DNA primase having the amino acid sequence of SEQ ID NO: 18; or
the Thermococcus celericrescens DNA primase having the amino acid sequence of SEQ ID NO: 20;
   or a functionally active fragment and/or variant thereof:
having at least 70% sequence identity with the amino acid sequence of any one of SEQ ID NOs: 2 to 13, 15, 16, 18 or 20; and
being capable of template-independent terminal nucleotidyl transferase activity; and
being capable of ab-initio single-stranded nucleic acid synthesis activity.

In one embodiment, said primase domain of an archaeal DNA primase belonging to the primase-polymerase family is the primase domain of:
the Thermococcus nautili sp. 30-1 DNA primase having the amino acid sequence of any one of SEQ ID NOs: 2 to 5; or
the Thermococcus sp. CIR10 DNA primase having the amino acid sequence of SEQ ID NO: 15; or
the Thermococcus peptonophilus DNA primase having the amino acid sequence of SEQ ID NO: 18; or
the Thermococcus celericrescens DNA primase having the amino acid sequence of SEQ ID NO: 20;
   or a functionally active fragment and/or variant thereof:
having at least 70% sequence identity with said amino acid sequence; and
being capable of ab-initio single-stranded nucleic acid synthesis activity; and
being capable of template-independent terminal nucleotidyl transferase activity.

In one embodiment, the nucleotide is immobilized onto a support.

In one embodiment, the ab-initio single-stranded nucleic acid synthesis is carried out at a temperature ranging from about 60° C. to about 95° C.

In one embodiment, said method is for ab-initio synthesis of nucleic acids with random nucleotide sequence, and the at least one nucleoside triphosphate does not comprise terminating nucleoside triphosphates.

In one embodiment, said method is for ab-initio sequence-controlled synthesis of nucleic acids, and the at least one nucleoside triphosphate is a terminating nucleoside triphosphate comprising a reversible 3′-blocking group.

In one embodiment, the method comprises the steps of:
a) providing the nucleotide with a free 3′-hydroxyl group;
b) contacting said nucleotide with a terminating nucleoside triphosphate in the presence of the primase domain of the archaeal DNA primase belonging to the primase-polymerase family or the functionally active fragment and/or variant thereof,
   thereby covalently binding said terminating nucleoside triphosphate to the free 3′-hydroxyl group of the nucleotide;
c) applying a washing solution to remove all reagents, in particular to remove unbound terminating nucleoside triphosphates;
d) cleaving the reversible 3′-blocking group of the covalently bound terminating nucleoside triphosphate in the presence of a cleaving agent;
   thereby obtaining a nucleotide with a free 3′-hydroxyl group;
e) optionally, applying a washing solution to remove all reagents, in particular to remove the cleaving agent;
f) optionally, reiterating steps b) to e) multiple times to synthetize the nucleic acid until desired length and nucleotide sequence.

In one embodiment, said method is for cleaning-up contaminating nucleoside triphosphates comprising a free 3′-hydroxyl group in a pool of terminating nucleoside triphosphates.

The present invention also relates to an isolated functionally active fragment of an archaeal DNA primase consisting of an amino acid sequence of any one of SEQ ID NOs: 3 to 13, 15, 16, 18 or 20, or a functionally active fragment and/or variant thereof:
having at least 70% sequence identity with said amino acid sequence; and
being capable of ab-initio single-stranded nucleic acid synthesis activity; and
being capable of template-independent terminal nucleotidyl transferase activity.

In one embodiment, the isolated functionally active fragment of the archaeal DNA primase or variant thereof consists of the amino acid sequence of any one of SEQ ID NOs: 3 to 13, 15, 16, 18 or 20.

In one embodiment, the isolated functionally active fragment of the archaeal DNA primase or variant thereof consists of an amino acid sequence of any one of SEQ ID NOs: 3 to 5, 15, 18 or 20, or a functionally active fragment and/or variant thereof:
having at least 70% sequence identity with said amino acid sequence; and
being capable of ab-initio single-stranded nucleic acid synthesis activity; and
being capable of template-independent terminal nucleotidyl transferase activity.

In one embodiment, the isolated functionally active fragment of the archaeal DNA primase or variant thereof consists of the amino acid sequence of any one of SEQ ID NOs: 3 to 5, 15, 18 or 20.

The present invention also relates to a nucleic acid encoding the functionally active fragment of an archaeal DNA primase according to the invention.

The present invention also relates to an expression vector comprising the nucleic acid according to the invention, operably linked to regulatory elements, preferably to a promoter.

The present invention also relates to a host cell comprising the expression vector according to the invention.

The present invention also relates to a method of producing the functionally active fragment of an archaeal DNA primase according to the invention, said method comprising:
(a) culturing the host cell according to the invention, under conditions suitable for the expression of said functionally active fragment of the archaeal DNA primase or variant thereof; and
(b) isolating said functionally active fragment of the archaeal DNA primase or variant thereof from said host cell.

The present invention also relates to a kit comprising:
a nucleotide with a free 3′-hydroxyl group, optionally immobilized onto a support;
at least one nucleoside triphosphate, optionally wherein the at least one nucleoside triphosphate is a terminating nucleoside triphosphate comprising a reversible 3′-blocking group; and
the isolated functionally active fragment of the archaeal DNA primase according to the invention.

### DETAILED DESCRIPTION

In a first aspect, the present invention relates to an isolated functionally active fragment of an archaeal DNA primase or variant thereof; a nucleic acid encoding the same; an expression vector comprising the latter; a host cell comprising this expression vector; and a method of production of said isolated functionally active fragment of an archaeal DNA primase or variant thereof.

“DNA primase” refer to enzymes involved in the replication of DNA, belonging to the class of RNA polymerases. They catalyze ab-initio synthesis of short RNA molecules called primers, typically from 4 to 15 nucleotides in length, from ribonucleoside triphosphates in the presence of a single stranded DNA template. DNA primase activity is required at the replication fork to initiate DNA synthesis by DNA polymerases (Frick & Richardson, 2001. Annu Rev Biochem. 70:39-80).

“Isolated” and any declensions thereof, as well as “purified” and any declensions thereof, are used interchangeably when with reference to an archaeal DNA primase or a functionally active fragment thereof, and mean that said archaeal DNA primase or functionally active fragment thereof is substantially free of other components (i.e., of contaminants) found in the natural environment in which said archaeal DNA primase or functionally active fragment thereof is normally found. Preferably, an isolated or purified archaeal DNA primase or functionally active fragment thereof is substantially free of other proteins or nucleic acids with which it is associated in a cell. By “substantially free”, it is meant that said isolated or purified archaeal DNA primase or functionally active fragment thereof represents more than 50% of a heterogeneous composition (i.e., is at least 50% pure), preferably, more than 60%, more than 70%, more than 80%, more than 90%, more than 95%, and more preferably still more than 98% or 99%. Purity can be evaluated by various methods known by the one skilled in the art, including, but not limited to, chromatography, gel electrophoresis, immunoassay, composition analysis, biological assay, and the like.

“Functionally active fragment”, with reference to an archaeal DNA primase, means a fragment or a domain of an archaeal DNA primase which is capable of ab-initio single-stranded nucleic acid synthesis activity, and preferably, of template-independent terminal nucleotidyl transferase activity. Means and methods to assess the activity of a fragment or a domain of an archaeal DNA primase are well known to the one skilled in the art. These include the assays described in the Example section of the present disclosure, and others, such as those described by Guilliam & Doherty (2017. Methods Enzymol. 591:327-353).

“Functionally active variant”, with reference to the primase domain of an archaeal DNA primase, means a protein which does not share 100% of sequence identity, but shares at least 70%, preferably at least 75%, 80%, 85%, 90%, 95%, 96%, 97%, 98%, 99% or more sequence identity, preferably of local sequence identity with the reference primase domain of an archaeal DNA primase, while maintaining its capacity of ab-initio single-stranded nucleic acid synthesis activity, and preferably also, of template-independent terminal nucleotidyl transferase activity. Means and methods to assess the activity of a variant of a primase domain of an archaeal DNA primase are well known to the one skilled in the art. These include the assays described in the Example section of the present disclosure, and others, such as those described by Guilliam & Doherty (2017. Methods Enzymol. 591:327-353).

In one embodiment, a functionally active fragment of an archaeal DNA primase or variant thereof is capable of ab-initio single-stranded nucleic acid synthesis activity. In one embodiment, a functionally active fragment of an archaeal DNA primase or variant thereof is capable of template-independent terminal nucleotidyl transferase activity. In one embodiment, a functionally active fragment of an archaeal DNA primase or variant thereof is capable of both ab-initio single-stranded nucleic acid synthesis activity and template-independent terminal nucleotidyl transferase activity.

By “ab-initio single-stranded nucleic acid synthesis activity” or “template-independent primase activity”, it is meant the synthesis of single stranded nucleic acid molecules in absence of both complementary nucleic acid template and initiator sequence, i.e., starting from a single nucleotide.

By “template-independent terminal nucleotidyl transferase activity”, it is meant the addition of nucleoside triphosphates to the 3′ terminus of a nucleic acid molecule, in absence of complementary nucleic acid template.

In one embodiment, the archaeal DNA primase belongs to the archaeo-eukaryotic primase (AEP) superfamily. In one embodiment, the archaeal DNA primase belongs to the primase-polymerase (prim-pol) family.

In one embodiment, the archaeal DNA primase is from an archaeon of the Thermococcus genus. The Thermococcus genus comprises several species among which, without limitations, Thermococcus acidaminovorans, Thermococcus aegaeus, Thermococcus aggregans, Thermococcus alcaliphilus, Thermococcus atlanticus, Thermococcus barophilus, Thermococcus barossii, Thermococcus celer, Thermococcus celericrescens, Thermococcus chitonophagus, Thermococcus cleftensis, Thermococcus coalescens, Thermococcus eurythermalis, Thermococcus fumicolans, Thermococcus gammatolerans, Thermococcus gorgonarius, Thermococcus guaymasensis, Thermococcus hydrothermalis, Thermococcus indicus, Thermococcus kodakarensis, Thermococcus litoralis, Thermococcus marinus, Thermococcus mexicalis, Thermococcus nautili, Thermococcus onnurineus, Thermococcus pacificus, Thermococcus paralvinellae, Thermococcus peptonophilus, Thermococcus piezophilus, Thermococcus prieurii, Thermococcus profundus, Thermococcus radiotolerans, Thermococcus sibiricus, Thermococcus siculi, Thermococcus stetteri, Thermococcus thioreducens, Thermococcus waimanguensis, Thermococcus waiotapuensis, and Thermococcus zilligii. The Thermococcus genus also comprises several unclassified strains among which, without limitation, Thermococcus sp. AEPII 1a, Thermococcus sp. 101 C5, Thermococcus sp. 11N.A5, Thermococcus sp. 12-4, Thermococcus sp. 13-2, Thermococcus sp. 13-3, Thermococcus sp. 1519, Thermococcus sp. 175, Thermococcus sp. 17S1, Thermococcus sp. 17S2, Thermococcus sp. 17S3, Thermococcus sp. 17S4, Thermococcus sp. 17S5, Thermococcus sp. 17S6, Thermococcus sp. 17S8, Thermococcus sp. 18S1, Thermococcus sp. 18S2, Thermococcus sp. 18S3, Thermococcus sp. 18S4, Thermococcus sp. 18S5, Thermococcus sp. 21-1, Thermococcus sp. 21S1, Thermococcus sp. 21S2, Thermococcus sp. 21S3, Thermococcus sp. 21S4, Thermococcus sp. 21S5, Thermococcus sp. 21S6, Thermococcus sp. 21S7, Thermococcus sp. 21S8, Thermococcus sp. 21S9, Thermococcus sp. 23-1, Thermococcus sp. 23-2, Thermococcus sp. 2319x1, Thermococcus sp. 26-2, Thermococcus sp. 26-3, Thermococcus sp. 26/2, Thermococcus sp. 28-1, Thermococcus sp. 29-1, Thermococcus sp. 300-Tc, Thermococcus sp. 31-1, Thermococcus sp. 31-3, Thermococcus sp. 40_45, Thermococcus sp. 4557, Thermococcus sp. 5-1, Thermococcus sp. 5-4, Thermococcus sp. 70-4-2, Thermococcus sp. 7324, Thermococcus sp. 83-5-2, Thermococcus sp. 9N2, Thermococcus sp. 9N2.20, Thermococcus sp. 9N2.21, Thermococcus sp. 9N3, Thermococcus sp. 9oN-7, Thermococcus sp. A4, Thermococcus sp. AF1T14.13, Thermococcus sp. AF1T1423, Thermococcus sp. AF1T20.11, Thermococcus sp. AF1T6.10, Thermococcus sp. AF1T6.12, Thermococcus sp. AF1T6.63, Thermococcus sp. AF2T511, Thermococcus sp. Ag85-vw, Thermococcus sp. AM4, Thermococcus sp. AMT11, Thermococcus sp. AMT7, Thermococcus sp. Anhete70478, Thermococcus sp. Anhete70-SCI, Thermococcus sp. Anhete85478, Thermococcus sp. Anhete85-SCI, Thermococcus sp. AT1273, Thermococcus sp. AV1, Thermococcus sp. AV2, Thermococcus sp. AV3, Thermococcus sp. AV6, Thermococcus sp. AV7, Thermococcus sp. AV9, Thermococcus sp. AV10, Thermococcus sp. AV11, Thermococcus sp. AV13, Thermococcus sp. AV14, Thermococcus sp. AV15, Thermococcus sp. AV16, Thermococcus sp. AV17, Thermococcus sp. AV18, Thermococcus sp. AV20, Thermococcus sp. AV21, Thermococcus sp. AV22, Thermococcus sp. Ax00-17, Thermococcus sp. Ax00-27, Thermococcus sp. Ax00-39, Thermococcus sp. Ax00-45, Thermococcus sp. Ax01-2, Thermococcus sp. Ax01-3, Thermococcus sp. Ax01-37, Thermococcus sp. Ax01-39, Thermococcus sp. Ax01-61, Thermococcus sp. Ax01-62, Thermococcus sp. Ax01-65, Thermococcus sp. Ax98-43, Thermococcus sp. Ax98-46, Thermococcus sp. Ax98-48, Thermococcus sp. Ax99-47, Thermococcus sp. Ax99-57, Thermococcus sp. Ax99-67, Thermococcus sp. AXTV6, Thermococcus sp. B1, Thermococcus sp. B1001, Thermococcus sp. B4, Thermococcus sp. BHI60a21, Thermococcus sp. BHI80a28, Thermococcus sp. BHI80a40, Thermococcus sp. Bubb.Bath, Thermococcus sp. BX13, Thermococcus sp. CAR-80, Thermococcus sp. Champagne, Thermococcus sp. CIR10, Thermococcus sp. CKU-1, Thermococcus sp. CKU-199, Thermococcus sp. CL2, Thermococcus sp. CMI, Thermococcus sp. CNR-5, Thermococcus sp. CX1, Thermococcus sp. CX2, Thermococcus sp. CX3, Thermococcus sp. CX4, Thermococcus sp. CYA, Thermococcus sp. Dex80a71, Thermococcus sp. Dex80a75, Thermococcus sp. DS-1, Thermococcus sp. DS1, Thermococcus sp. DT4, Thermococcus sp. ENR5, Thermococcus sp. EP1, Thermococcus sp. ES5, Thermococcus sp. ES6, Thermococcus sp. ES7, Thermococcus sp. ES8, Thermococcus sp. ES9, Thermococcus sp. ES10, Thermococcus sp. ES11, Thermococcus sp. ES12, Thermococcus sp. ES13, Thermococcus sp. EXT12c, Thermococcus sp. EXT9, Thermococcus sp. Fe85_1_2, Thermococcus sp. GB18, Thermococcus sp. GB20, Thermococcus sp. GE8, Thermococcus sp. Gorda2, Thermococcus sp. Gorda3, Thermococcus sp. Gorda4, Thermococcus sp. Gorda5, Thermococcus sp. Gorda6, Thermococcus sp. GR2, Thermococcus sp. GR4, Thermococcus sp. GR5, Thermococcus sp. GR6, Thermococcus sp. GR7, Thermococcus sp. GT, Thermococcus sp. GU5L5, Thermococcus sp. HJ21, Thermococcus sp. IRI33, Thermococcus sp. IRI35c, Thermococcus sp. IRI48, Thermococcus sp. JCM 11816, Thermococcus sp. JDF-3, Thermococcus sp. JdF3, Thermococcus sp. JdFR-02, Thermococcus sp. KBA1, Thermococcus sp. KI, Thermococcus sp. KS-8, Thermococcus sp. LMO-A1, Thermococcus sp. LMO-A2, Thermococcus sp. LMO-A3, Thermococcus sp. LMO-A4, Thermococcus sp. LMO-A5, Thermococcus sp. LMO-A6, Thermococcus sp. LMO-A7, Thermococcus sp. LMO-A8, Thermococcus sp. LMO-A9, Thermococcus sp. LS1, Thermococcus sp. LS2, Thermococcus sp. M36, Thermococcus sp. M39, Thermococcus sp. MA2.27, Thermococcus sp. MA2.28, Thermococcus sp. MA2.29, Thermococcus sp. MA2.33, Thermococcus sp. MAR1, Thermococcus sp. MAR2, Thermococcus sp. MCR132, Thermococcus sp. MCR133, Thermococcus sp. MCR134, Thermococcus sp. MCR135, Thermococcus sp. MCR175, Thermococcus sp. MV1, Thermococcus sp. MV2, Thermococcus sp. MV3, Thermococcus sp. MVS, Thermococcus sp. MV10, Thermococcus sp. MV11, Thermococcus sp. MV12, Thermococcus sp. MV13, Thermococcus sp. MV1031, Thermococcus sp. MV1049, Thermococcus sp. MV1083, Thermococcus sp. MV1092, Thermococcus sp. MV1099, Thermococcus sp. MZ1, Thermococcus sp. MZ2, Thermococcus sp. MZ3, Thermococcus sp. MZ5, Thermococcus sp. MZ6, Thermococcus sp. MZ7, Thermococcus sp. MZ8, Thermococcus sp. MZ9, Thermococcus sp. MZ10, Thermococcus sp. MZ11, Thermococcus sp. MZ12, Thermococcus sp. MZ13, Thermococcus sp. NS85-T, Thermococcus sp. P6, Thermococcus sp. Pd70, Thermococcus sp. Pd85, Thermococcus sp. PK, Thermococcus sp. PK(2011), Thermococcus sp. Rt3, Thermococcus sp. SB611, Thermococcus sp. SN531, Thermococcus sp. SRB55_1, Thermococcus sp. SRB70_1, Thermococcus sp. SRB70_10, Thermococcus sp. SY113, Thermococcus sp. Tc-1-70, Thermococcus sp. Tc-1-85, Thermococcus sp. Tc-1-95, Thermococcus sp. Tc-2-85, Thermococcus sp. Tc-2-95, Thermococcus sp. Tc-365-70, Thermococcus sp. Tc-365-85, Thermococcus sp. Tc-365-95, Thermococcus sp. Tc-4-70, Thermococcus sp. Tc-4-85, Thermococcus sp. Tc-I-70, Thermococcus sp. Tc-I-85, Thermococcus sp. Tc-S-70, Thermococcus sp. Tc-S-85, Thermococcus sp. Tc55_1, Thermococcus sp. Tc55_12, Thermococcus sp. Tc70-4C-I, Thermococcus sp. Tc70-4C-S, Thermococcus sp. Tc70-7C-I, Thermococcus sp. Tc70-7C-S, Thermococcus sp. Tc70-CRC-I, Thermococcus sp. Tc70-CRC-S, Thermococcus sp. Tc70-MC-S, Thermococcus sp. Tc70-SC-I, Thermococcus sp. Tc70-SC-S, Thermococcus sp. Tc70-vw, Thermococcus sp. Tc70_1, Thermococcus sp. Tc70_10, Thermococcus sp. Tc70_11, Thermococcus sp. Tc70_12, Thermococcus sp. Tc70_20, Thermococcus sp. Tc70_6, Thermococcus sp. Tc70_9, Thermococcus sp. Tc85-0 age SC, Thermococcus sp. Tc85-4C-I, Thermococcus sp. Tc85-4C-S, Thermococcus sp. Tc85-7C-S, Thermococcus sp. Tc85-CRC-I, Thermococcus sp. Tc85-CRC-S, Thermococcus sp. Tc85-MC-I, Thermococcus sp. Tc85-MC-S, Thermococcus sp. Tc85-SC-I, Thermococcus sp. Tc85-SC-ISCS, Thermococcus sp. Tc85-SC-S, Thermococcus sp. Tc85_1, Thermococcus sp. Tc85_10, Thermococcus sp. Tc85_11, Thermococcus sp. Tc85_12, Thermococcus sp. Tc85_13, Thermococcus sp. Tc85_19, Thermococcus sp. Tc85_2, Thermococcus sp. Tc85_20, Thermococcus sp. Tc85_9, Thermococcus sp. Tc95-CRC-I, Thermococcus sp. Tc95-CRC-S, Thermococcus sp. Tc95-MC-I, Thermococcus sp. Tc95-MC-S, Thermococcus sp. Tc95-SC-S, Thermococcus sp. TK1, Thermococcus sp. TKM 55-W7-A, Thermococcus sp. TM1, Thermococcus sp. TP-33, Thermococcus sp. TP-37, Thermococcus sp. TS3, Thermococcus sp. TVG2, and Thermococcus sp. vp197.

In one embodiment, the archaeal DNA primase is selected from the group consisting of Thermococcus nautili sp. 30-1 DNA primase, Thermococcus sp. CIR10 DNA primase, Thermococcus peptonophilus DNA primase, and Thermococcus celericrescens DNA primase; or a functionally active fragment and/or variant thereof.

In one embodiment, the archaeal DNA primase is Thermococcus nautili sp. 30-1 primase; or a functionally active fragment and/or variant thereof.

In one embodiment, the amino acid sequence of the Thermococcus nautili sp. 30-1 DNA primase comprises or consists of SEQ ID NO: 1, which represents the amino acid sequence of the protein “tn2-12p” from Thermococcus nautili sp. 30-1 with NCBI Reference Sequence WP_013087990 version 1 of 2019-05-01.

SEQ ID NO: 1
MSSLRPSSIIIDIYKVIQDHPDSSRWAIELRFLPKPISSEWIFITDIEE
RASEIDKVLTKYNIMKKKDAYVSMAIHDFEKVTAKLKRVQEEENKKATE
GERRLRRITLDKIQGDSETTSGFTLALVVDIDNTKIHDTRIIENEEEAF
EASKREWEALKPKLQELGFLPRWILYTGGGLQLWFVSDKLEPISVIDRA
SEIIPNIMNGVNGVKGLLSEGFKADNIFDPARIVRAPLTFNHKYRTIIK
DEDGTERVVPTQVKGRVIEFNDVRISLTEFLDRLEAYAKEKGIPLEKPT
KRKFLELASKRYEVTSSNFEALAERLFTELRPWWEIAKEKGWSRHHLTM
GIATYILRNTNLTPEQLIGSENSPGLWELVFAKLVEAGLEDPDDWKNRA
STIRDAYKKIESGKKVATKAYLRKYIEGLSEEDAVQILLSVKRALLPYL
KAVDVKRISKYSARPYEITEEIPKSWEDIDENRKKATGVWYIDFLALET
ANDIYFEDLPKPPVFYIRFVEKNKEKFKLNETLYHSFLTWLGITEGEPL
DRTELIDLLVEKFGFTVEDLKAIYYRKILTLLKPEGMRTPKCIQEFLFE
LATEGNLPEDKIRHLAHWVKFYARPLRHSTTSVLLKGRGKPVDMRLAIW
AKVVEFFAEDDEVAEELITTFKKAYMGAEPPFPCIGAESCPFYPDHRAC
PFIVPKRKEVLPVSIVDVQLHGSDGIVVLVGGPTEVTAFTLEGKVEWIK
TTKKTVKYPIAEWFLDRFAKEYLSLPEAPSWWKLEEVTEILKSRARVVK
SQFDKFEDYLEQFIEWLQKENSRRGILPYEKADENHLFIKGEWVGIPPG
FAREFYSGELLIGGPTFRRMLEQKLGKDYRKMSAKIHLNTGLKDKRNCY
FVSVEWFRKHVGEPNIQEITSEGDVSFDGLSYDDEEEGVVG

In one embodiment, the amino acid sequence of a functionally active fragment of the Thermococcus nautili sp. 30-1 DNA primase (herein termed “PolpTN2_{Δ311-923}”) is as set forth in SEQ ID NO: 2.

SEQ ID NO: 2
MSSLRPSSIIIDIYKVIQDHPDSSRWAIELRFLPKPISSEWIFITDIEE
RASEIDKVLTKYNIMKKKDAYVSMAIHDFEKVTAKLKRVQEEENKKATE
GERRLRRITLDKIQGDSETTSGFTLALVVDIDNTKIHDTRIIENEEEAF
EASKREWEALKPKLQELGFLPRWILYTGGGLQLWFVSDKLEPISVIDRA
SEIIPNIMNGVNGVKGLLSEGFKADNIFDPARIVRAPLTFNHKYRTIIK
DEDGTERVVPTQVKGRVIEFNDVRISLTEFLDRLEAYAKEKGIPLEKPT
KRKFLELASKRYEVTS

In one embodiment, the amino acid sequence of a functionally active fragment of the Thermococcus nautili sp. 30-1 DNA primase (herein termed “PolpTN2_{Δ90-96Δ311-923}”) is as set forth in SEQ ID NO: 3.

SEQ ID NO: 3
MSSLRPSSIIIDIYKVIQDHPDSSRWAIELRFLPKPISSEWIFITDIEE
RASEIDKVLTKYNIMKKKDAYVSMAIHDFEKVTAKLKRVQETEGERRLR
RITLDKIQGDSETTSGFTLALVVDIDNTKIHDTRIIENEEEAFEASKRE
WEALKPKLQELGFLPRWILYTGGGLQLWFVSDKLEPISVIDRASEIIPN
IMNGVNGVKGLLSEGFKADNIFDPARIVRAPLTFNHKYRTIIKDEDGTE
RVVPTQVKGRVIEFNDVRISLTEFLDRLEAYAKEKGIPLEKPTKRKFLE
LASKRYEVTS

In one embodiment, the amino acid sequence of a functionally active fragment of the Thermococcus nautili sp. 30-1 DNA primase (herein termed “PolpTN2_{Δ205-211Δ311-923}”) is as set forth in SEQ ID NO: 4.

SEQ ID NO: 4
MSSLRPSSIIIDIYKVIQDHPDSSRWAIELRFLPKPISSEWIFITDIEE
RASEIDKVLTKYNIMKKKDAYVSMAIHDFEKVTAKLKRVQEEENKKATE
GERRLRRITLDKIQGDSETTSGFTLALVVDIDNTKIHDTRIIENEEEAF
EASKREWEALKPKLQELGFLPRWILYTGGGLQLWFVSDKLEPISVIDRA
SEIIPNIMNGLLSEGFKADNIFDPARIVRAPLTFNHKYRTIIKDEDGTE
RVVPTQVKGRVIEFNDVRISLTEFLDRLEAYAKEKGIPLEKPTKRKFLE
LASKRYEVTS

In one embodiment, the amino acid sequence of a functionally active fragment of the Thermococcus nautili sp. 30-1 DNA primase (herein termed “PolpTN2_{Δ248-254Δ311-923}”) is as set forth in SEQ ID NO: 5.

SEQ ID NO: 5
MSSLRPSSIIIDIYKVIQDHPDSSRWAIELRFLPKPISSEWIFITDIEE
RASEIDKVLTKYNIMKKKDAYVSMAIHDFEKVTAKLKRVQEEENKKATE
GERRLRRITLDKIQGDSETTSGFTLALVVDIDNTKIHDTRIIENEEEAF
EASKREWEALKPKLQELGFLPRWILYTGGGLQLWFVSDKLEPISVIDRA
SEIIPNIMNGVNGVKGLLSEGFKADNIFDPARIVRAPLTFNHKYRTIIK
DEDPTQVKGRVIEFNDVRISLTEFLDRLEAYAKEKGIPLEKPTKRKFLE
LASKRYEVTS

In one embodiment, the amino acid sequence of a functionally active fragment of the Thermococcus nautili sp. 30-1 DNA primase (herein termed “PolpTN2_{Δ243-254Δ311-923}”) is as set forth in SEQ ID NO: 6.

SEQ ID NO: 6
MSSLRPSSIIIDIYKVIQDHPDSSRWAIELRFLPKPISSEWIFITDIEE
RASEIDKVLTKYNIMKKKDAYVSMAIHDFEKVTAKLKRVQEEENKKATE
GERRLRRITLDKIQGDSETTSGFTLALVVDIDNTKIHDTRIIENEEEAF
EASKREWEALKPKLQELGFLPRWILYTGGGLQLWFVSDKLEPISVIDRA
SEIIPNIMNGVNGVKGLLSEGFKADNIFDPARIVRAPLTFNHKYRTPTQ
VKGRVIEFNDVRISLTEFLDRLEAYAKEKGIPLEKPTKRKFLELASKRY
EVTS

In one embodiment, the amino acid sequence of a functionally active fragment of the Thermococcus nautili sp. 30-1 DNA primase (herein termed “PolpTN2_{Δ90-96Δ205-211Δ1311-923}”) is as set forth in SEQ ID NO: 7.

SEQ ID NO: 7
MSSLRPSSIIIDIYKVIQDHPDSSRWAIELRFLPKPISSEWIFITDIEE
RASEIDKVLTKYNIMKKKDAYVSMAIHDFEKVTAKLKRVQETEGERRLR
RITLDKIQGDSETTSGFTLALVVDIDNTKIHDTRIIENEEEAFEASKRE
WEALKPKLQELGFLPRWILYTGGGLQLWFVSDKLEPISVIDRASEIIPN
IMNGLLSEGFKADNIFDPARIVRAPLTFNHKYRTIIKDEDGTERVVPTQ
VKGRVIEFNDVRISLTEFLDRLEAYAKEKGIPLEKPTKRKFLELASKRY
EVTS

In one embodiment, the amino acid sequence of a functionally active fragment of the Thermococcus nautili sp. 30-1 DNA primase (herein termed “PolpTN2_{Δ90-96Δ248-254Δ311-923}”) is as set forth in SEQ ID NO: 8.

SEQ ID NO: 8
MSSLRPSSIIIDIYKVIQDHPDSSRWAIELRFLPKPISSEWIFITDIEE
RASEIDKVLTKYNIMKKKDAYVSMAIHDFEKVTAKLKRVQETEGERRLR
RITLDKIQGDSETTSGFTLALVVDIDNTKIHDTRIIENEEEAFEASKRE
WEALKPKLQELGFLPRWILYTGGGLQLWFVSDKLEPISVIDRASEIIPN
IMNGVNGVKGLLSEGFKADNIFDPARIVRAPLTFNHKYRTIIKDEDPTQ
VKGRVIEFNDVRISLTEFLDRLEAYAKEKGIPLEKPTKRKFLELASKRY
EVTS

In one embodiment, the amino acid sequence of a functionally active fragment of the Thermococcus nautili sp. 30-1 DNA primase (herein termed “PolpTN2_{Δ90-96Δ243-254Δ311-923}”) is as set forth in SEQ ID NO: 9.

SEQ ID NO: 9
MSSLRPSSIIIDIYKVIQDHPDSSRWAIELRFLPKPISSEWIFITDIEE
RASEIDKVLTKYNIMKKKDAYVSMAIHDFEKVTAKLKRVQETEGERRLR
RITLDKIQGDSETTSGFTLALVVDIDNTKIHDTRIIENEEEAFEASKRE
WEALKPKLQELGFLPRWILYTGGGLQLWFVSDKLEPISVIDRASEIIPN
IMNGVNGVKGLLSEGFKADNIFDPARIVRAPLTFNHKYRTPTQVKGRVI
EFNDVRISLTEFLDRLEAYAKEKGIPLEKPTKRKFLELASKRYEVTS

In one embodiment, the amino acid sequence of a functionally active fragment of the Thermococcus nautili sp. 30-1 DNA primase (herein termed “PolpTN2_{Δ205-211Δ248-254Δ311-923}”) is as set forth in SEQ ID NO: 10.

SEQ ID NO: 10
MSSLRPSSIIIDIYKVIQDHPDSSRWAIELRFLPKPISSEWIFITDIEE
RASEIDKVLTKYNIMKKKDAYVSMAIHDFEKVTAKLKRVQEEENKKATE
GERRLRRITLDKIQGDSETTSGFTLALVVDIDNTKIHDTRIIENEEEAF
EASKREWEALKPKLQELGFLPRWILYTGGGLQLWFVSDKLEPISVIDRA
SEIIPNIMNGLLSEGFKADNIFDPARIVRAPLTFNHKYRTIIKDEDPTQ
VKGRVIEFNDVRISLTEFLDRLEAYAKEKGIPLEKPTKRKFLELASKRY
EVTS

In one embodiment, the amino acid sequence of a functionally active fragment of the Thermococcus nautili sp. 30-1 DNA primase (herein termed “PolpTN2_{Δ205-211Δ243-254Δ311-923}”) is as set forth in SEQ ID NO: 11.

SEQ ID NO: 11
MSSLRPSSIIIDIYKVIQDHPDSSRWAIELRFLPKPISSEWIFITDIEE
RASEIDKVLTKYNIMKKKDAYVSMAIHDFEKVTAKLKRVQEEENKKATE
GERRLRRITLDKIQGDSETTSGFTLALVVDIDNTKIHDTRIIENEEEAF
EASKREWEALKPKLQELGFLPRWILYTGGGLQLWFVSDKLEPISVIDRA
SEIIPNIMNGLLSEGFKADNIFDPARIVRAPLTFNHKYRTPTQVKGRVI
EFNDVRISLTEFLDRLEAYAKEKGIPLEKPTKRKFLELASKRYEVTS

In one embodiment, the amino acid sequence of a functionally active fragment of the Thermococcus nautili sp. 30-1 DNA primase (herein termed “PolpTN2_{Δ90-96Δ205-211Δ248-254Δ311-923}”) is as set forth in SEQ ID NO: 12.

SEQ ID NO: 12
MSSLRPSSIIIDIYKVIQDHPDSSRWAIELRFLPKPISSEWIFITDIEE
RASEIDKVLTKYNIMKKKDAYVSMAIHDFEKVTAKLKRVQETEGERRLR
RITLDKIQGDSETTSGFTLALVVDIDNTKIHDTRIIENEEEAFEASKRE
WEALKPKLQELGFLPRWILYTGGGLQLWFVSDKLEPISVIDRASEIIPN
IMNGLLSEGFKADNIFDPARIVRAPLTFNHKYRTIIKDEDPTQVKGRVI
EFNDVRISLTEFLDRLEAYAKEKGIPLEKPTKRKFLELASKRYEVTS

In one embodiment, the amino acid sequence of a functionally active fragment of the Thermococcus nautili sp. 30-1 DNA primase (herein termed “PolpTN2_{Δ90-96Δ205-211Δ243-254Δ311-923}”) is as set forth in SEQ ID NO: 13.

SEQ ID NO: 13
MSSLRPSSIIIDIYKVIQDHPDSSRWAIELRFLPKPISSEWIFITDIEE
RASEIDKVLTKYNIMKKKDAYVSMAIHDFEKVTAKLKRVQETEGERRLR
RITLDKIQGDSETTSGFTLALVVDIDNTKIHDTRIIENEEEAFEASKRE
WEALKPKLQELGFLPRWILYTGGGLQLWFVSDKLEPISVIDRASEIIPN
IMNGLLSEGFKADNIFDPARIVRAPLTFNHKYRTPTQVKGRVIEFNDVR
ISLTEFLDRLEAYAKEKGIPLEKPTKRKFLELASKRYEVTS

In one embodiment, the amino acid sequence of the Thermococcus sp. CIR10 DNA primase comprises or consists of SEQ ID NO: 14, which represents the amino acid sequence of the protein “primase/polymerase” from Thermococcus sp. CIR10 with NCBI Reference Sequence WP_015243587 version 1 of 2016-06-18.

SEQ ID NO: 14
MSGREFKRPSDVIIDIYKVIQDHPEAGRLAIEFRFYPYPTSEWILLNDI
EDKAREIDKVLFKNNILGKKEAYISMAIHDFDEVTKKLEKLQELEHEKA
QKEGRQPKEITLRHVQGEATGKIHTTVSSYTLTLVVDIDVNEIHDSKAV
ESEEKALEVSKRAWEVLKPNLEELGIKPRYVFFTGGGIQLWFVAPEPEN
ISVIDKAAEIIPPVLNTLLPEGYSVDNIFDRARIVRVPFTVNYKYKTPD
GKPLELRGRLLEFNDVRTPLGDILEKLEAYAKGHKISLGSTSRSGKFRG
VAGRYEVKKENFEELAKRLVEELAPWFKKIKERGGSRHHLVNAIAAYIA
RNTNLTEEDLLGKDQEDGTHVVGLWELVHSKLVELGLEDPGDWSNRYHT
IKDVYEKLYAGTTLGTRAYMMKYLNVSEEEAIEILRSVKRALFPYLHPV
NVQVISKFEAKPYSKEEAPTEWEAVDEDRKKAVGIWYIEVLALETANYV
YIEDLSKPGVFYIVEKVKRTVKVGKKEKGVEVDEYHFNPALWQSFLNWL
GIKEGEPIEREELWNLLLEKFDIKDYELRAIYFRKILHLLSPEGMRRPR
CVEEFLRELADEGFLSEDKVRHLAHWIKFYAKPLRHSTTSIMLRAKGKP
VDMRMAVWAKVVEFFAEDEETAQGLIETFREAYEQAEPPFPCFGARECP
FFQEHRGCPFIAPKRDEILAVSLVDVQLHGSDGIVIIVGSEEGTKKFVH
KGKVEWQKQGKSKIKYPVAEWFLDVYAKEFLSLPEAPSWSHEEVTEILK
SRARVVRSQLNEFDEYFDNFIDWLRSENARGIYPYEKADSSHIFIKGNM
IGIPPRLAEDFYRNELGISGRKFKEMLIRELGSYYLGKKAAWIKLSSGQ
HNGVNCYFISLDWFKKIVGEPNIKDIEAEGDIGSGGFNYEEEEGEA

In one embodiment, the amino acid sequence of a functionally active fragment of the Thermococcus sp. CIR10 DNA primase (herein termed “PolpCIR10_{Δ303-928}”) is as set forth in SEQ ID NO: 15.

SEQ ID NO: 15
MSGREFKRPSDVIIDIYKVIQDHPEAGRLAIEFRFYPYPTSEWILLNDI
EDKAREIDKVLFKNNILGKKEAYISMAIHDFDEVTKKLEKLQELEHEKA
QKEGRQPKEITLRHVQGEATGKIHTTVSSYTLTLVVDIDVNEIHDSKAV
ESEEKALEVSKRAWEVLKPNLEELGIKPRYVFFTGGGIQLWFVAPEPEN
ISVIDKAAEIIPPVLNTLLPEGYSVDNIFDRARIVRVPFTVNYKYKTPD
GKPLELRGRLLEFNDVRTPLGDILEKLEAYAKGHKISLGSTSRSGKFRG
VAGRYEVKK

In one embodiment, the amino acid sequence of a functionally active fragment of the Thermococcus sp. CIR10 DNA primase (herein termed “PolpCIR10_{Δ93-98Δ303-928}”) is as set forth in SEQ ID NO: 16.

SEQ ID NO: 16
MSGREFKRPSDVIIDIYKVIQDHPEAGRLAIEFRFYPYPTSEWILLNDI
EDKAREIDKVLFKNNILGKKEAYISMAIHDFDEVTKKLEKLQEQKEGRQ
PKEITLRHVQGEATGKIHTTVSSYTLTLVVDIDVNEIHDSKAVESEEKA
LEVSKRAWEVLKPNLEELGIKPRYVFFTGGGIQLWFVAPEPENISVIDK
AAEIIPPVLNTLLPEGYSVDNIFDRARIVRVPFTVNYKYKTPDGKPLEL
RGRLLEFNDVRTPLGDILEKLEAYAKGHKISLGSTSRSGKFRGVAGRYE
VKK

In one embodiment, the amino acid sequence of the Thermococcus peptonophilus DNA primase comprises or consists of SEQ ID NO: 17, which represents the amino acid sequence of an “hypothetical protein” from Thermococcus peptonophilus with NCBI Reference Sequence WP_062389070 version 1 of 2016-03-28.

SEQ ID NO: 17
MSELTPGKVLADVYKVIQDHPEAGRLAIELRFYPVIKSEWVLLNDIEDK
ARDIDKVLAKQNLIKGKEAYVSMAIHGFEAVKKKLEKLRESVEEGKVRK
LGLENVQGEAKGKVHPTVSNYTLTLVVDVDIEAVHKLKVVEDVDKVFEK
AKEGWLALKPVFEELGVLPRYVFFTGGGLQLWFVAPKLEDIAVIDRASG
IVPNVLNALLPEGFVVDNIFDRARIVRAPLTVNHKYKAPNGARVGVKGR
LIEFNDVRVSLSEVLDKLEVYAKERGIQLGGQEKVRGGRGFVNVRYVVK
KEELETLALNLADELIPWFKKVKERGGSWHHLVNAIGAYVVRNTNLSLE
DLIGKDNPDGTHVVGLWEIVFQRLVEKSAEDPGDWVNRRNTIKDVYEKH
IAGKPLGTRAYLKKYLPVSDEEVVEILMAVRRALLPFLKEVKKVDSSGF
GVAPYKKTAPRSWDEVDEDRKRATGRWYVWKLAFNTAEYLFTDELPKAG
TFYIDVWMKEGKREWMKRFFNEALFRSFIEDGLGYKYGAPVEREELFER
LVEVFNITDEEVRGIYIDAALSLLSPVGMRTPPCIEEFIMEFAANGSLS
EDKVRHLARWIKLYAKPLKHSTTTTKLVGAGYKVDMRMAVWAKLVEFFA
EDDEVARELVRVFKEEYGAAEPPFTCIGTKTCQFYLNEKMCPFIIPKEK
EILAVSLIDVQRHESDGLVVIVGGDKEVRTFVKKGNVEWVKKTERREKY
PVAEWFIDVFATEYLSVSPDDLDVDLEEVTDILKSRARVVKSRLNELED
MYEKFVEWLKRENAVRGVLPYEKADFNHLFIKGNMIGIPPALAEEFYRF
ELNIKGSEFREMLEKKLGFHYTKKAVKLSVGEKKDVRRCYLVSLEWFRK
VVGEPNVKDVVMAGDIALSGLVYYESGEEVVE

In one embodiment, the amino acid sequence of a functionally active fragment of the Thermococcus peptonophilus DNA primase (herein termed “PolpTpep_{Δ295-914}”) is as set forth in SEQ ID NO: 18.

SEQ ID NO: 18
MSELTPGKVLADVYKVIQDHPEAGRLAIELRFYPVIKSEWVLLNDIEDK
ARDIDKVLAKQNLIKGKEAYVSMAIHGFEAVKKKLEKLRESVEEGKVRK
LGLENVQGEAKGKVHPTVSNYTLTLVVDVDIEAVHKLKVVEDVDKVFEK
AKEGWLALKPVFEELGVLPRYVFFTGGGLQLWFVAPKLEDIAVIDRASG
IVPNVLNALLPEGFVVDNIFDRARIVRAPLTVNHKYKAPNGARVGVKGR
LIEFNDVRVSLSEVLDKLEVYAKERGIQLGGQEKVRGGRGFVNVRYVVK
K

In one embodiment, the amino acid sequence of the Thermococcus celericrescens DNA primase comprises or consists of SEQ ID NO: 19, which represents the amino acid sequence of an “hypothetical protein” from Thermococcus celericrescens with NCBI Reference Sequence WP_058937716 version 1 of 2016-01-06.

SEQ ID NO: 19
MSELTPGKVLADVYKVIQDHPEAGRLAIELRFYPVIKSEWVLLNDIEDK
ARDIDKVLAKKNIINGKEAYVSMAIHDFGAVKKKLEKLREKAEGERARR
IGLENVQGEAKGKVHPTVSNYTLALVVDIDIEEVHKSRVVEDVEAVFER
AKKGWLALRPVFEELGVLPRYVFFTGGGLQIWFVAPELEDIAVIDRASG
IVPGVLNALLPEGFVVDNIFDRARIVRAPLTVNHKYKAPNGAGLGVKGR
LIEFNDVRVSLSEVLDKLEAYAKEKGIQLGGQERASGVRVFGKVRYEVK
KERLETLALNLADELAPWFKKVKERGGSWHHLVNAIGAYIVRNTNLSLE
DLIGKDNPDGTHVVGLWELVFQRLVEKGAEDPSDWLNRRNTIKDVYEKH
IAGKPLGTRAYLKKYLPVSDEEVVEILMAARRALLPFLKGVKRAGSSGF
GVFPYKDTAPRSWNEVEAERKRATGLWYVWSLAFSTAEYIFTDELSKAG
TFFILVKEGKSIKSVFNETLFRSFIEEGLGYKYGMPVRRDELFERLVEV
FNITDEEVRGVYIDAALSLLSPVGMRTPPCIEEFIMEFAANGNLPEDKV
RHLARWIKLYAKPLKHSTTTTKLVGAGYNVDMRMAVWAKLVEFFAEDDE
VAGELVRIFKEEYKEAEPPFTCIGARTCPFYLKDDVAKMCPFIFPKEKE
ILAVSLIDVQRHESDGIVILVGGAREVRTFIKKGKVEWVKRTKKTEKYP
IAEWFIDVFATEYLGVPPDNLDFDLKDVTGILKSRERVVPSQLNEVEDW
YEKFVEWLKRENEVRGVLPYKKADVHHLFIKDNMIGIPPALAEEFYRYE
ANIKPSEFREMLEVKLSIHYKKKAVKLSVGEKKDVRRCYLVSLEWFRKV
VGEPNVKDVVMAGDIALSGLVYYESGEEWE

In one embodiment, the amino acid sequence of a functionally active fragment of the Thermococcus celericrescens DNA primase (herein termed “PolpTcele_{Δ295-913}”) is as set forth in SEQ ID NO: 20.

SEQ ID NO: 20
MSELTPGKVLADVYKVIQDHPEAGRLAIELRFYPVIKSEWVLLNDIEDK
ARDIDKVLAKKNIINGKEAYVSMAIHDFGAVKKKLEKLREKAEGERARR
IGLENVQGEAKGKVHPTVSNYTLALVVDIDIEEVHKSRVVEDVEAVFER
AKKGWLALRPVFEELGVLPRYVFFTGGGLQIWFVAPELEDIAVIDRASG
IVPGVLNALLPEGFVVDNIFDRARIVRAPLTVNHKYKAPNGAGLGVKGR
LIEFNDVRVSLSEVLDKLEAYAKEKGIQLGGQERASGVRVFGKVRYEVK
K

In one embodiment, the isolated functionally active fragment of an archaeal DNA primase or variant thereof according to the present invention comprises or consists of an amino acid sequence selected from the group comprising or consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 15, SEQ ID NO: 16, SEQ ID NO: 18, and SEQ ID NO: 20, or a fragment and/or variant thereof. In one embodiment, the isolated functionally active fragment of an archaeal DNA primase or variant thereof according to the present invention does not consist of an amino acid sequence selected from the group comprising or consisting of SEQ ID NO: 1, SEQ ID NO: 14, SEQ ID NO: 17 and SEQ ID NO: 19.

In one embodiment, the isolated functionally active fragment of an archaeal DNA primase or variant thereof according to the present invention comprises or consists of an amino acid sequence selected from the group comprising or consisting of SEQ ID NO: 2, SEQ ID NO: 15, SEQ ID NO: 18, and SEQ ID NO: 20, or a fragment and/or variant thereof. In one embodiment, the isolated functionally active fragment of an archaeal DNA primase or variant thereof according to the present invention does not consist of an amino acid sequence selected from the group comprising or consisting of SEQ ID NO: 1, SEQ ID NO: 14, SEQ ID NO: 17 and SEQ ID NO: 19.

In one embodiment, the isolated functionally active fragment of an archaeal DNA primase or variant thereof according to the present invention comprises or consists of an amino acid sequence selected from the group comprising or consisting of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 16, SEQ ID NO: 18, and SEQ ID NO: 20, or a fragment and/or variant thereof. In one embodiment, the isolated functionally active fragment of an archaeal DNA primase or variant thereof according to the present invention does not consist of an amino acid sequence selected from the group comprising or consisting of SEQ ID NO: 1, SEQ ID NO: 14, SEQ ID NO: 17 and SEQ ID NO: 19.

In one embodiment, the isolated functionally active fragment of an archaeal DNA primase or variant thereof according to the present invention comprises or consists of an amino acid sequence selected from the group comprising or consisting of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, and SEQ ID NO: 13, or a fragment and/or variant thereof. In one embodiment, the isolated functionally active fragment of an archaeal DNA primase or variant thereof according to the present invention does not consist of the amino acid sequence of SEQ ID NO: 1.

In one embodiment, the isolated functionally active fragment of an archaeal DNA primase or variant thereof according to the present invention comprises or consists of an amino acid sequence selected from the group comprising or consisting of SEQ ID NO: 3, SEQ ID NO: 4, and SEQ ID NO: 5, or a fragment and/or variant thereof. In one embodiment, the isolated functionally active fragment of an archaeal DNA primase or variant thereof according to the present invention does not consist of the amino acid sequence of SEQ ID NO: 1.

In one embodiment, the isolated functionally active fragment of an archaeal DNA primase or variant thereof according to the present invention comprises or consists of an amino acid sequence selected from the group comprising or consisting of SEQ ID NO: 15, SEQ ID NO: 18, and SEQ ID NO: 20, or a fragment and/or variant thereof. In one embodiment, the isolated functionally active fragment of an archaeal DNA primase or variant thereof according to the present invention does not consist of an amino acid sequence selected from the group comprising or consisting of SEQ ID NO: 14, SEQ ID NO: 17 and SEQ ID NO: 19.

In one embodiment, a fragment of the isolated functionally active fragment of an archaeal DNA primase or variant thereof according to the present invention comprises or consists of at least 50% contiguous amino acid residues of said isolated functionally active fragment of an archaeal DNA primase or variant thereof, preferably at least 60%, 70%, 80%, 90%, 95% or more contiguous amino acid residues of said isolated functionally active fragment of an archaeal DNA primase or variant thereof.

In one embodiment, a fragment of the isolated functionally active fragment of an archaeal DNA primase or variant thereof according to the present invention remains capable of ab-initio single-stranded nucleic acid synthesis activity, and preferably, of template-independent terminal nucleotidyl transferase activity.

In one embodiment, a variant of the isolated functionally active fragment of an archaeal DNA primase or fragment thereof according to the present invention shares at least 70%, preferably at least 75%, 80%, 85%, 90%, 95%, 96%, 97%, 98%, 99% or more sequence identity, preferably local sequence identity with said isolated functionally active fragment of an archaeal DNA primase or fragment thereof.

Sequence identity refers to the number of identical or similar amino acids in a comparison between a test and a reference sequence. Sequence identity can be determined by sequence alignment of protein sequences to identify regions of similarity or identity. For purposes herein, sequence identity is generally determined by alignment to identify identical residues. The alignment can be local or global. Matches, mismatches and gaps can be identified between compared sequences. Gaps are null amino acids inserted between the residues of aligned sequences so that identical or similar characters are aligned. Generally, there can be internal and terminal gaps. When using gap penalties, sequence identity can be determined with no penalty for end gaps (e.g., terminal gaps are not penalized). Alternatively, sequence identity can be determined without taking into account gaps as

$$\frac{number of identical positions}{length of the total aligned sequence} \times 100 .$$

A global alignment is an alignment that aligns two sequences from beginning to end, aligning each letter in each sequence only once. An alignment is produced, regardless of whether or not there is similarity or identity between the sequences. For example, 50% sequence identity based on global alignment means that in an alignment of the full sequence of two compared sequences, each of 100 nucleotides in length, 50% of the residues are the same. It is understood that global alignment can also be used in determining sequence identity even when the length of the aligned sequences is not the same. The differences in the terminal ends of the sequences will be taken into account in determining sequence identity, unless the “no penalty for end gaps” is selected. Generally, a global alignment is used on sequences that share significant similarity over most of their length. Exemplary algorithms for performing global alignment include the Needleman-Wunsch algorithm (Needleman & Wunsch, 1970. J Mol Biol. 48(3):443-53). Exemplary programs and software for performing global alignment are publicly available and include the Global Sequence Alignment Tool available at the National Center for Biotechnology Information (NCBI) website (http://ncbi.nlm.nih.gov), and the program available at deepc2.psi.iastate.edu/aat/align/align.html.

A local alignment is an alignment that aligns two sequences, but only aligns those portions of the sequences that share similarity or identity. Hence, a local alignment determines if sub-segments of one sequence are present in another sequence. If there is no similarity, no alignment will be returned. Local alignment algorithms include BLAST or Smith-Waterman algorithm (Smith & Waterman, 1981. Adv Appl Math. 2(4):482-9). For example, 50% sequence identity based on local alignment means that in an alignment of the full sequence of two compared sequences of any length, a region of similarity or identity of 100 nucleotides in length has 50% of the residues that are the same in the region of similarity or identity.

For purposes herein, sequence identity can be determined by standard alignment algorithm programs used with default gap penalties established by each supplier. Default parameters for the GAP program can include:
(1) a unary comparison matrix (containing a value of 1 for identities and 0 for non-identities) and the weighted comparison matrix of Gribskov & Burgess (1986. Nucleic Acids Res. 14(16):6745-63), as described by Schwartz & Dayhoff (1979. Matrices for detecting distant relationships. In Dayhoff (Ed.), Atlas of protein sequences. 5:353-358. Washington, D.C.: National Biomedical Research Foundation);
(2) a penalty of 3.0 for each gap and an additional 0.10 penalty for each symbol in each gap; and
(3) no penalty for end gaps.

Whether any sequence of a functionally active fragment of an archaeal DNA primase or fragment thereof, and a variant of this sequence, have amino acid sequences that are at least 70%, preferably at least 75%, 80%, 85%, 90%, 95%, 96%, 97%, 98%, 99% or more “identical”, or other similar variations reciting a percent identity, can be determined using known computer algorithms based on local or global alignment (see, e.g., https://en.wikipedia.org/wiki/List_of_sequence_alignment_software, providing links to dozens of known and publicly available alignment databases and programs).

Generally, for purposes herein, sequence identity is determined using computer algorithms based on global alignment, such as the Needleman-Wunsch Global Sequence Alignment tool available from NCBI/BLAST (http://blast.ncbi.nlm.nih.gov/Blast.cgi); or LAlign (William Pearson implementing the Huang and Miller algorithm [Huang & Miller, 1991. Adv Appl Math. 12(3):337-57).

Typically, the full-length sequence of each of the compared functionally active fragments of archaeal DNA primases or fragments thereof is aligned across the full-length of each sequence in a global alignment. Local alignment also can be used when the sequences being compared are substantially the same length.

Therefore, the term identity represents a comparison or alignment between a test (the variant) and a reference sequence (the functionally active fragment of an archaeal DNA primase or fragment thereof). In one exemplary embodiment, “at least 70% of sequence identity” refers to percent identities from 70 to 100% relative to the reference sequence. Identity at a level of 70% or more is indicative of the fact that, assuming for exemplification purposes a test and reference sequence length of 100 amino acids are compared, no more than 30 out of 100 amino acids in the test sequence differ from those of the reference sequence. Such differences can be represented as point mutations randomly distributed over the entire length of an amino acid sequence or they can be clustered in one or more locations of varying length up to the maximum allowable, e.g., 30/100 amino acid difference (approximately 70% identity). Differences can also be due to deletions or truncations of amino acid residues. Differences are defined as amino acid substitutions, insertions or deletions. Depending on the length of the compared sequences, at the level of homologies or identities above about 85-90%, the result can be independent of the program and gap parameters set; such high levels of identity can be assessed readily, often without relying on software.

Also encompassed herein are isolated functionally active fragment of an archaeal DNA primase or variant thereof according to the present invention, fused to a processivity factor.

By “processivity factor”, it is meant a polypeptide domain or subdomain which confers sequence-independent nucleic acid interactions, and is associated with the isolated functionally active fragment of an archaeal DNA primase or fragment thereof according to the present invention by covalent or noncovalent interactions. Processivity factors may confer a lower dissociation constant between the archaeal DNA primase and the nucleic acid substrate, allowing for more nucleotide incorporations on average before dissociation of the archaeal DNA primase from the substrate or initiator sequence.

Processivity factors function by multiple sequence-independent nucleic acid binding mechanisms: the primary mechanism is electrostatic interaction between the nucleic acid phosphate backbone and the processivity factor; the second is steric interactions between the processivity factor and the minor groove structure of a nucleic acid duplex; the third mechanism is topological restraint, where interactions with the nucleic acid are facilitated by clamp proteins that completely encircle the nucleic acid, with which they associate.

Exemplary sequence-independent nucleic acid binding domains are known in the art, and are traditionally classified according to the preferred nucleic acid substrate, e.g., DNA or RNA and strandedness, such as single-stranded or double-stranded.

Various polypeptide domains have been identified as nucleic acid binders. These polypeptide domains include four general structural topologies known to bind single-stranded DNA: oligonucleotide-binding (OB) folds, K homology (KH) domains, RNA recognition motifs (RRMs), and whirly domains, as described in Dickey et al., 2013. Structure. 21(7):1074-1084.

Oligonucleotide-binding domains (OBDs) are exemplary DNA binding domains structurally conserved in multiple DNA processing proteins. OBDs bind with single-stranded DNA ligands from 3 to 11 nucleotides per OB fold and dissociation constants ranging from low-picomolar to high-micromolar levels. Affinities roughly correlate with the length of single-stranded DNA bound. Some OBDs may confer sequence specific binding, while others are non-sequence specific. Exemplary OBD containing DNA-binding proteins specifically bind single-stranded DNA are so called “single-stranded DNA binding proteins” or “SSBs”. SSB domains are well known to those skilled in the art, as described, e.g., in Keck (Ed.), 2016. Single-stranded DNA binding proteins (Vol. 922, Methods in Molecular Biology). Totowa, N.J.: Humana Press; and Shereda et al., 2008. Crit Rev Biochem Mol Biol. 43(5):289-318. SSBs describe a family of evolved molecular chaperones of single-stranded DNA.

Several exemplary prokaryotic SSBs have been characterized as known to those skilled in the art. These SSBs include, but are not limited to; Escherichia coli SSB (see, e.g., Raghunathan et al., 2000. Nat Struct Biol. 7(8):648-652), Deinococcus radiodurans SSB (see, e.g., Lockhart & DeVeaux, 2013. PLoS One. 8(8):e71651), Sulfolobus solfataricus SSB (see, e.g., Paytubi et al., 2012. Proc Natl Acad Sci USA. 109(7):E398-E405), Thermus thermophillus SSB and Thermus aquaticus SSB (see, e.g., Witte et al., 2008. Biophys J. 94(6):2269-2279), and Deinococcus radiopugnans SSB (see, e.g., Filipkowski et al., 2006. Extremophiles. 10(6):607-614).

In non-eubacterial systems, functional eukaryotic homologs to the prokaryotic SSB protein family are known to those skilled in the art. Replication protein A (RPA) is an exemplary homolog used in DNA replication, recombination and DNA repair in eukaryotes. The RPA heterotrimer is comprised of RPA70, RPA32, RPA14 subunits as described in Iftode et al., 1999. Crit Rev Biochem Mol Biol. 34(3):141-180.

The present invention also relates to a nucleic acid encoding the isolated functionally active fragment of the archaeal DNA primase or variant thereof described above.

It also relates to an expression vector comprising the nucleic acid encoding the isolated functionally active fragment of the archaeal DNA primase or variant thereof described above.

The term “expression vector” refers to a recombinant DNA molecule containing the desired coding nucleic acid sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

It also relates to a host cell comprising the expression vector comprising the nucleic acid encoding the isolated functionally active fragment of the archaeal DNA primase or variant thereof described above.

It also relates to a method of producing and purifying the isolated functionally active fragment of the archaeal DNA primase or variant thereof described above.

In one embodiment, the method comprises:
culturing a host cell comprising the expression vector comprising the nucleic acid encoding the isolated functionally active fragment of the archaeal DNA primase or variant thereof described above, under conditions suitable for the expression of said functionally active fragment of the archaeal DNA primase or variant thereof, and
isolating the functionally active fragment of the archaeal DNA primase or variant thereof from said host cell.

This recombinant process can be used for large scale production of the functionally active fragment of the archaeal DNA primase or variant thereof.

In one embodiment, the expressed functionally active fragment of the archaeal DNA primase or variant thereof is further purified.

In a second aspect, the present invention relates to a method for ab-initio single-stranded nucleic acid synthesis, comprising contacting the 3′-hydroxyl group of a nucleotide with at least one nucleoside triphosphate (or a combination of nucleoside triphosphates) in the presence of an archaeal DNA primase or a functionally active fragment and/or variant thereof, thereby covalently binding said nucleoside triphosphate to the 3′-hydroxyl group of the nucleotide.

In one embodiment, the method of the present invention is a method for ab-initio single-stranded nucleic acid synthesis with random nucleotide sequence. In one embodiment, the method of the present invention is a method for ab-initio, sequence-controlled single-stranded nucleic acid synthesis of nucleic acids.

References to a “nucleic acid” synthesis method include methods of synthesizing lengths of DNA (deoxyribonucleic acid), RNA (ribonucleic acid), or mixes thereof, wherein a first nucleotide (n) is coupled with at least one further nucleotide (n+1), thereby obtaining at least a dimer of nucleotides. The term “nucleic acid” also encompasses nucleic acid analogues, such as, without limitation, xeno nucleic acids (XNA), which are synthetic nucleic acid analogues that have a different sugar backbone and/or outgoing motif than the natural DNAs and RNAs. The term “nucleic acid” hence also encompasses mixed XNA/DNA, mixed XNA/RNA and mixed XNA/DNA/RNA. Examples of XNAs include those described in Schmidt, 2010. Bioessays. 32(4):322-331 and Nie et al., 2020. Molecules. 25(15):E3483, the content of which is herein incorporated by reference. Some examples include, but are not limited to, 1,5-anhydrohexitol nucleic acid (HNA), cyclohexene nucleic acid (CeNA), threose nucleic acid (TNA), glycol nucleic acid (GNA), locked nucleic acid (LNA), peptide nucleic acid (PNA), and fluoro arabino nucleic acid (FANA) (Schmidt, 2008. Syst Synth Biol. 2(1-2):1-6; Ran et al., 2009. Nat Nanotechnol. 4(10):6; Kershner et al., 2009. Nat Nanotechnol. 4(9):557-61; Marliere, 2009. Syst Synth Biol. 3(1-4):77-84; Tones et al., 2003. Microbiology. 149(Pt 12):3595-601; Vastmans et al., 2001. Nucleic Acids Res. 29(15):3154-63; Ichida et al., 2005. Nucleic Acids Res. 33(16):5219-25; Kempeneers et al., 2005. Nucleic Acids Res. 33(12):3828-36; Loakes et al., 2009. J Am Chem Soc. 131(41):14827-37).

References to a “sequence-controlled” nucleic acid synthesis method illustrate those methods of nucleic acid synthesis which allow the specific addition of at least one nucleotide (n+1) to a first nucleotide (n), i.e., the synthesized nucleic acid has a defined—by contrast to random—nucleotide sequence.

In one embodiment, the archaeal DNA primase or the functionally active fragment and/or variant thereof belongs to the archaeo-eukaryotic primase (AEP) superfamily.

In one embodiment, the archaeal DNA primase or the functionally active fragment and/or variant thereof is from an archaeon of the Thermococcales order.

In one embodiment, the archaeal DNA primase is from an archaeon of the Thermococcus genus.

In one embodiment, the archaeal DNA primase or the functionally active fragment and/or variant thereof belongs to the primase-polymerase (prim-pol) family (also termed “PolpTN2-like family” by Kazlauskas et al., 2018. J Mol Biol. 430(5):737-750).

In one embodiment, the archaeal DNA primase or the functionally active fragment and/or variant thereof comprises or consists of the primase domain of an archaeal DNA primase belonging to the primase-polymerase (prim-pol) family (as shown by Kazlauskas et al., 2018. J Mol Biol. 430(5):737-750 in their FIG. 6).

In one embodiment, the archaeal DNA primase is selected from the group consisting of Thermococcus nautili sp. 30-1 DNA primase, Thermococcus sp. CIR10 DNA primase, Thermococcus peptonophilus DNA primase, and Thermococcus celericrescens DNA primase; or a functionally active fragment and/or variant thereof, as described hereinabove.

In one embodiment, the archaeal DNA primase is Thermococcus nautili sp. 30-1 DNA primase; or a functionally active fragment and/or variant thereof, as described hereinabove.

In one embodiment, the amino acid sequence of the Thermococcus nautili sp. 30-1 DNA primase comprises or consists of SEQ ID NO: 1, as described hereinabove.

In one embodiment, the amino acid sequence of a functionally active fragment of the Thermococcus nautili sp. 30-1 DNA primase is selected from the group comprising or consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, and SEQ ID NO: 13.

In one embodiment, the amino acid sequence of a functionally active fragment of the Thermococcus nautili sp. 30-1 DNA primase is selected from the group comprising or consisting of SEQ ID NO: 2, SEQ ID NO: 3, SEQ ID NO: 4, and SEQ ID NO: 5.

In one embodiment, the amino acid sequence of a functionally active fragment of the Thermococcus nautili sp. 30-1 DNA primase (herein termed “PolpTN2_{Δ311-923}”) is as set forth in SEQ ID NO: 2.

In one embodiment, the amino acid sequence of a functionally active fragment of the Thermococcus nautili sp. 30-1 DNA primase (herein termed “PolpTN2_{Δ90-96Δ311-923}”) is as set forth in SEQ ID NO: 3.

In one embodiment, the amino acid sequence of a functionally active fragment of the Thermococcus nautili sp. 30-1 DNA primase (herein termed “PolpTN2_{Δ205-211Δ311-923}”) is as set forth in SEQ ID NO: 4.

In one embodiment, the amino acid sequence of a functionally active fragment of the Thermococcus nautili sp. 30-1 DNA primase (herein termed “PolpTN2_{Δ248-254Δ311-923}”) is as set forth in SEQ ID NO: 5.

In one embodiment, the amino acid sequence of a functionally active fragment of the Thermococcus nautili sp. 30-1 DNA primase (herein termed “PolpTN2_{Δ243-254Δ311-923}”) is as set forth in SEQ ID NO: 6.

In one embodiment, the amino acid sequence of a functionally active fragment of the Thermococcus nautili sp. 30-1 DNA primase (herein termed “PolpTN2_{Δ90-96Δ205-211Δ311-923}”) is as set forth in SEQ ID NO: 7.

In one embodiment, the amino acid sequence of a functionally active fragment of the Thermococcus nautili sp. 30-1 DNA primase (herein termed “PolpTN2_{Δ90-96Δ248-254Δ311-923}”) is as set forth in SEQ ID NO: 8.

In one embodiment, the amino acid sequence of a functionally active fragment of the Thermococcus nautili sp. 30-1 DNA primase (herein termed “PolpTN2_{Δ90-96Δ243-254Δ311-923}”) is as set forth in SEQ ID NO: 9.

In one embodiment, the amino acid sequence of a functionally active fragment of the Thermococcus nautili sp. 30-1 DNA primase (herein termed “PolpTN2_{Δ205-211Δ248-254Δ311-923}”) is as set forth in SEQ ID NO: 10.

In one embodiment, the amino acid sequence of a functionally active fragment of the Thermococcus nautili sp. 30-1 DNA primase (herein termed “PolpTN2_{Δ205-211Δ243-254Δ311-923}”) is as set forth in SEQ ID NO: 11.

In one embodiment, the amino acid sequence of a functionally active fragment of the Thermococcus nautili sp. 30-1 DNA primase (herein termed “PolpTN2_{Δ90-96Δ205-211Δ248-254Δ311-923}”) is as set forth in SEQ ID NO: 12.

In one embodiment, the amino acid sequence of a functionally active fragment of the Thermococcus nautili sp. 30-1 DNA primase (herein termed “PolpTN2_{Δ90-96Δ205-211Δ243-254Δ311-923}”) is as set forth in SEQ ID NO: 13.

In one embodiment, the amino acid sequence of the Thermococcus sp. CIR10 DNA primase comprises or consists of SEQ ID NO: 14, as described hereinabove.

In one embodiment, the amino acid sequence of a functionally active fragment of the Thermococcus sp. CIR10 DNA primase (herein termed “PolpCIR10_{Δ303-928}”) is as set forth in SEQ ID NO: 15.

In one embodiment, the amino acid sequence of a functionally active fragment of the Thermococcus sp. CIR10 DNA primase (herein termed “PolpCIR10_{Δ93-98Δ303-928}”) is as set forth in SEQ ID NO: 16.

In one embodiment, the amino acid sequence of the Thermococcus peptonophilus DNA primase comprises or consists of SEQ ID NO: 17, as described hereinabove.

In one embodiment, the amino acid sequence of a functionally active fragment of the Thermococcus peptonophilus DNA primase (herein termed “PolpTpep_{Δ295-914}”) is as set forth in SEQ ID NO: 18.

In one embodiment, the amino acid sequence of the Thermococcus celericrescens DNA primase comprises or consists of SEQ ID NO: 19, as described hereinabove.

In one embodiment, the amino acid sequence of a functionally active fragment of the Thermococcus celericrescens DNA primase (herein termed “PolpTcele_{Δ295-913}”) is as set forth in SEQ ID NO: 20.

In one embodiment, the archaeal DNA primase or functionally active fragment and/or variant thereof comprises or consists of an amino acid sequence selected from the group comprising or consisting of SEQ ID NO: 1, SEQ ID NO: 14, SEQ ID NO: 17 and SEQ ID NO: 19; or a functionally active fragment and/or variant thereof.

In one embodiment, the archaeal DNA primase or functionally active fragment and/or variant thereof comprises or consists of an amino acid sequence selected from the group comprising or consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 15, SEQ ID NO: 16, SEQ ID NO: 18, and SEQ ID NO: 20; or a functionally active fragment and/or variant thereof.

In one embodiment, the archaeal DNA primase or functionally active fragment and/or variant thereof comprises or consists of an amino acid sequence selected from the group comprising or consisting of SEQ ID NO: 2, SEQ ID NO: 15, SEQ ID NO: 18 and SEQ ID NO: 20; or a functionally active fragment and/or variant thereof.

In one embodiment, the archaeal DNA primase or functionally active fragment and/or variant thereof comprises or consists of the amino acid sequence set forth in SEQ ID NO: 1; or a functionally active fragment and/or variant thereof. In one embodiment, the archaeal DNA primase or functionally active fragment and/or variant thereof comprises or consists of the amino acid sequence set forth in SEQ ID NO: 2; or a functionally active fragment and/or variant thereof.

In one embodiment, the archaeal DNA primase or functionally active fragment and/or variant thereof comprises or consists of the amino acid sequence set forth in SEQ ID NO: 1; or a functionally active fragment and/or variant thereof. In one embodiment, the archaeal DNA primase or functionally active fragment and/or variant thereof comprises or consists of the amino acid sequence set forth in SEQ ID NO: 3; or a functionally active fragment and/or variant thereof.

In one embodiment, the archaeal DNA primase or functionally active fragment and/or variant thereof comprises or consists of the amino acid sequence set forth in SEQ ID NO: 1; or a functionally active fragment and/or variant thereof. In one embodiment, the archaeal DNA primase or functionally active fragment and/or variant thereof comprises or consists of the amino acid sequence set forth in SEQ ID NO: 4; or a functionally active fragment and/or variant thereof.

In one embodiment, the archaeal DNA primase or functionally active fragment and/or variant thereof comprises or consists of the amino acid sequence set forth in SEQ ID NO: 1; or a functionally active fragment and/or variant thereof. In one embodiment, the archaeal DNA primase or functionally active fragment and/or variant thereof comprises or consists of the amino acid sequence set forth in SEQ ID NO: 5; or a functionally active fragment and/or variant thereof.

In one embodiment, the archaeal DNA primase or functionally active fragment and/or variant thereof comprises or consists of the amino acid sequence set forth in SEQ ID NO: 1; or a functionally active fragment and/or variant thereof. In one embodiment, the archaeal DNA primase or functionally active fragment and/or variant thereof comprises or consists of the amino acid sequence set forth in SEQ ID NO: 6; or a functionally active fragment and/or variant thereof.

In one embodiment, the archaeal DNA primase or functionally active fragment and/or variant thereof comprises or consists of the amino acid sequence set forth in SEQ ID NO: 1; or a functionally active fragment and/or variant thereof. In one embodiment, the archaeal DNA primase or functionally active fragment and/or variant thereof comprises or consists of the amino acid sequence set forth in SEQ ID NO: 7; or a functionally active fragment and/or variant thereof.

In one embodiment, the archaeal DNA primase or functionally active fragment and/or variant thereof comprises or consists of the amino acid sequence set forth in SEQ ID NO: 1; or a functionally active fragment and/or variant thereof. In one embodiment, the archaeal DNA primase or functionally active fragment and/or variant thereof comprises or consists of the amino acid sequence set forth in SEQ ID NO: 8; or a functionally active fragment and/or variant thereof.

In one embodiment, the archaeal DNA primase or functionally active fragment and/or variant thereof comprises or consists of the amino acid sequence set forth in SEQ ID NO: 1; or a functionally active fragment and/or variant thereof. In one embodiment, the archaeal DNA primase or functionally active fragment and/or variant thereof comprises or consists of the amino acid sequence set forth in SEQ ID NO: 9; or a functionally active fragment and/or variant thereof.

In one embodiment, the archaeal DNA primase or functionally active fragment and/or variant thereof comprises or consists of the amino acid sequence set forth in SEQ ID NO: 1; or a functionally active fragment and/or variant thereof. In one embodiment, the archaeal DNA primase or functionally active fragment and/or variant thereof comprises or consists of the amino acid sequence set forth in SEQ ID NO: 10; or a functionally active fragment and/or variant thereof.

In one embodiment, the archaeal DNA primase or functionally active fragment and/or variant thereof comprises or consists of the amino acid sequence set forth in SEQ ID NO: 1; or a functionally active fragment and/or variant thereof. In one embodiment, the archaeal DNA primase or functionally active fragment and/or variant thereof comprises or consists of the amino acid sequence set forth in SEQ ID NO: 11; or a functionally active fragment and/or variant thereof.

In one embodiment, the archaeal DNA primase or functionally active fragment and/or variant thereof comprises or consists of the amino acid sequence set forth in SEQ ID NO: 1; or a functionally active fragment and/or variant thereof. In one embodiment, the archaeal DNA primase or functionally active fragment and/or variant thereof comprises or consists of the amino acid sequence set forth in SEQ ID NO: 12; or a functionally active fragment and/or variant thereof.

In one embodiment, the archaeal DNA primase or functionally active fragment and/or variant thereof comprises or consists of the amino acid sequence set forth in SEQ ID NO: 1; or a functionally active fragment and/or variant thereof. In one embodiment, the archaeal DNA primase or functionally active fragment and/or variant thereof comprises or consists of the amino acid sequence set forth in SEQ ID NO: 13; or a functionally active fragment and/or variant thereof.

In one embodiment, the archaeal DNA primase or functionally active fragment and/or variant thereof comprises or consists of the amino acid sequence set forth in SEQ ID NO: 14; or a functionally active fragment and/or variant thereof. In one embodiment, the archaeal DNA primase or functionally active fragment and/or variant thereof comprises or consists of the amino acid sequence set forth in SEQ ID NO: 15; or a functionally active fragment and/or variant thereof.

In one embodiment, the archaeal DNA primase or functionally active fragment and/or variant thereof comprises or consists of the amino acid sequence set forth in SEQ ID NO: 14; or a functionally active fragment and/or variant thereof. In one embodiment, the archaeal DNA primase or functionally active fragment and/or variant thereof comprises or consists of the amino acid sequence set forth in SEQ ID NO: 16; or a functionally active fragment and/or variant thereof.

In one embodiment, the archaeal DNA primase or functionally active fragment and/or variant thereof comprises or consists of the amino acid sequence set forth in SEQ ID NO: 17; or a functionally active fragment and/or variant thereof. In one embodiment, the archaeal DNA primase or functionally active fragment and/or variant thereof comprises or consists of the amino acid sequence set forth in SEQ ID NO: 18; or a functionally active fragment and/or variant thereof.

In one embodiment, the archaeal DNA primase or functionally active fragment and/or variant thereof comprises or consists of the amino acid sequence set forth in SEQ ID NO: 19; or a functionally active fragment and/or variant thereof. In one embodiment, the archaeal DNA primase or functionally active fragment and/or variant thereof comprises or consists of the amino acid sequence set forth in SEQ ID NO: 20; or a functionally active fragment and/or variant thereof.

In one embodiment, a fragment of the archaeal DNA primase or functionally active fragment and/or variant thereof comprises or consists of at least 50% of contiguous amino acid residues of said archaeal DNA primase or functionally active fragment and/or variant thereof, preferably at least 60%, 70%, 80%, 90%, 95% or more of contiguous amino acid residues of said archaeal DNA primase or functionally active fragment and/or variant thereof.

In one embodiment, a fragment of the archaeal DNA primase or functionally active fragment and/or variant thereof remains capable of both ab-initio single-stranded nucleic acid synthesis activity and of template-independent terminal nucleotidyl transferase activity.

In one embodiment, the archaeal DNA primase or the functionally active fragment and/or variant thereof is fused to a processivity factor.

Processivity factors have been described hereinabove, which description applies mutatis mutandis to the archaeal DNA primase or the functionally active fragment and/or variant thereof.

In one embodiment, the nucleotide is a single nucleotide. In other words, the nucleotide is not a 3′-end nucleotide of an initiator sequence.

In one embodiment, the method for ab-initio single-stranded nucleic acid synthesis does not comprise contacting the 3′-hydroxyl group of an initiator sequence with at least one nucleoside triphosphate (or a combination of nucleoside triphosphates).

By “initiator sequence” or “primer”, it is meant a short oligonucleotide with a free 3′-end onto which a nucleoside triphosphate could be covalently bound, i.e., the nucleic acid would be synthesized from the 3′-end of the initiator sequence.

One skilled in the art will readily understand that the method of the present invention allows the synthesis of single stranded nucleic acid molecules, starting from a single nucleotide. This strictly applies to the first round of ab-initio single-stranded nucleic acid synthesis, yielding a nucleic acid molecule comprising 2 nucleotides. However, the method described herein can further be reiterated to allow the addition of further nucleoside triphosphates to the synthetized nucleic acid molecule (i.e., through the template-independent terminal nucleotidyl transferase activity of the archaeal DNA primase or the functionally active fragment and/or variant thereof).

In one embodiment, the nucleotide may be immobilized onto a support. In particular, the use of supports allows to easily filter, wash and/or elute reagents and by-products, without washing away the synthesized nucleic acid.

Suitable examples of supports include, but are not limited to, beads, slides, chips, particles, strands, gels, sheets, tubing, spheres, containers, capillaries, pads, slices, films, culture dishes, microtiter plates, and the like. Exemplary materials that can be used for such supports include, but are not limited to, acrylics, carbon (e.g., graphite, carbon-fiber), cellulose (e.g., cellulose acetate), ceramics, controlled-pore glass, cross-linked polysaccharides (e.g., agarose, SEPHAROSE™ or alginate), gels, glass (e.g., modified or functionalized glass), gold (e.g., atomically smooth Au(111)), graphite, inorganic glasses, inorganic polymers, latex, metal oxides (e.g., SiO₂, TiO₂, stainless steel), metalloids, metals (e.g., atomically smooth Au(111)), mica, molybdenum sulfides, nanomaterials (e.g., highly oriented pyrolitic graphite (HOPG) nanosheets), nitrocellulose, NYLON™, optical fiber bundles, organic polymers, paper, plastics, polacryloylmorpholide, poly(4-methylbutene), polyethylene terephthalate), poly(vinyl butyrate), polybutylene, polydimethylsiloxane (PDMS), polyethylene, polyformaldehyde, polymethacrylate, polypropylene, polysaccharides, polystyrene, polyurethanes, polyvinylidene difluoride (PVDF), quartz, rayon, resins, rubbers, semiconductor material, silica, silicon (e.g., surface-oxidized silicon), sulfide, and TEFLON™; or a mixture thereof.

In one embodiment, the nucleotide is immobilized onto a support via a reversible interacting moiety, such as, e.g., a chemically-cleavable linker, an enzymatically-cleavable linker, or any other suitable means.

It is thus conceivable that the synthetized nucleic acid be ultimately cleaved from the support and, e.g., amplified using an appropriate pair of forward and reverse primer sequences, complementary to the synthetized nucleic acid.

Additionally, or alternatively, the immobilized nucleotide may be a uridine.

It is thus conceivable that the synthetized nucleic acid be ultimately cleaved from the support using (1) an uracil-DNA glycosylase (UDG) to generate an abasic site, and (2) an apurinic/apyrimidinic (AP) site endonuclease to cleave the synthetized nucleic acid at the abasic site.

By “nucleoside triphosphate” or “NTP”, it is referred herein to a molecule containing a nitrogenous base bound to a 5-carbon sugar (typically, either ribose or deoxyribose), with three phosphate groups bound to the sugar at position 5. The term “nucleoside triphosphate” also encompasses nucleoside triphosphate analogues, such as, nucleoside triphosphates with a different sugar and/or a different nitrogenous base than the natural NTPs, as well as nucleoside triphosphates with a modified 2′-OH, 3′-OH and/or 5′-triphosphate positions. In particular, nucleoside triphosphate analogues include those useful for the synthesis of xeno nucleic acids (XNA), as defined hereinabove. Non-limiting examples of such synthetic nucleoside triphosphate analogues are given in FIG. 4 of Chakravarthy et al., 2017 (Theranostics. 7(16):3933-3947), the content of which Figure is herein incorporated by reference. Further non-limiting examples of such synthetic nucleoside triphosphate analogues are given in [0250] to [0280] of US 2009-0286696, the content of which paragraphs is herein incorporated by reference.

A nucleoside triphosphate containing a deoxyribose is typically referred to as deoxynucleoside triphosphate and abbreviated as dNTP. Consistently, a nucleoside triphosphate containing a ribose is typically referred to as ribonucleoside triphosphate and abbreviated as rNTP.

Examples of deoxynucleoside triphosphates include, but are not limited to, deoxyadenosine triphosphate (dATP), deoxyguanosine triphosphate (dGTP), deoxycytidine triphosphate (dCTP), and deoxythymidine triphosphate (dTTP). Further examples of deoxynucleoside triphosphates include deoxyuridine triphosphate (dUTP), deoxyinosine triphosphate (dITP), and deoxyxanthosine triphosphate (dXTP).

Examples of ribonucleoside triphosphates include, but are not limited to, adenosine triphosphate (ATP), guanosine triphosphate (GTP), cytidine triphosphate (CTP) and uridine triphosphate (UTP). Further examples of nucleoside triphosphates include N⁶-methyladenosine triphosphate (m⁶ATP), 5-methyluridine triphosphate (m⁵UTP), 5-methylcytidine triphosphate (m⁵CTP), pseudouridine triphosphate (ψUTP), inosine triphosphate (ITP), xanthosine triphosphate (XTP), and wybutosine triphosphate (yWTP).

Other types of nucleosides may be bound to three phosphates to form nucleoside triphosphates, such as naturally occurring modified nucleosides and artificial nucleosides.

In one embodiment, the at least one nucleoside triphosphate is a selected nucleoside triphosphate. In one embodiment, the at least one nucleoside triphosphate is a combination of (optionally, selected) nucleoside triphosphates.

By “selected” with reference to nucleoside triphosphates, it is meant a nucleoside triphosphate or a combination of nucleoside triphosphates purposely chosen among the various possibilities of nucleoside triphosphates, including, but not limited to those described above, with the idea of synthetizing either (1) a nucleic acid with a random sequence or (2) a nucleic acid with a defined nucleotide sequence.

By “combination of nucleoside triphosphates”, it is meant a mix of at least two different nucleoside triphosphates.

In one embodiment, the method of the present invention is a method for ab-initio single-stranded nucleic acid synthesis with random nucleotide sequence, which comprises contacting the free 3′-hydroxyl group of a nucleotide with a combination of (optionally, selected) nucleoside triphosphates in the presence of an archaeal DNA primase or a functionally active fragment and/or variant thereof, thereby covalently and randomly binding said combination of (optionally, selected) nucleoside triphosphates to the 3′-hydroxyl group of the nucleotide.

In this embodiment, the (optionally, selected) combination of nucleoside triphosphates does not comprise terminating nucleoside triphosphates.

In one embodiment, the method of the present invention is a method for ab-initio single-stranded, sequence-controlled nucleic acid synthesis, which comprises contacting the 3′-hydroxyl group of a nucleotide with a selected nucleoside triphosphate in the presence of an archaeal DNA primase or a functionally active fragment and/or variant thereof, thereby covalently binding said selected nucleoside triphosphate to the 3′-hydroxyl group of the nucleotide.

In the latter embodiment of sequence-controlled synthesis of nucleic acids, the 3′-hydroxyl group of a nucleotide is contacted with a selected terminating nucleoside triphosphate.

By “terminating nucleoside triphosphate”, also sometimes termed “3′-blocked nucleoside triphosphates” or “3′-protected nucleoside triphosphates”, it is referred to nucleoside triphosphates which have an additional group (hereafter, “3′-blocking group” or “3′-protecting group”) on their 3′-end (i.e., at position 3 of their 5-carbon sugar), for the purpose of preventing further, undesired, addition of nucleoside triphosphates after specific addition of the selected nucleotide (n+1) to the nucleotide (n).

In one embodiment, the 3′-blocking group may be reversible (can be removed from the nucleoside triphosphate) or irreversible (cannot be removed from the nucleoside triphosphate), i.e., the terminating nucleoside triphosphate may be a reversible terminating nucleoside triphosphate or a non-reversible terminating nucleoside triphosphate.

In one embodiment, the 3′-blocking group is reversible, and removal of the 3′-blocking group from the nucleoside triphosphate (e.g., using a cleaving agent) allows the addition of further nucleoside triphosphate to the synthetized nucleic acid.

Examples of reversible 3′-blocking groups include, but are not limited to, methyl, methoxy, oxime, 2-nitrobenzyl, 2-cyanoethyl, allyl, amine, aminoxy, azidomethyl, tert-butoxy ethoxy (TBE), propargyl, acetyl, quinone, coumarin, aminophenol derivative, ketal, N-methyl-anthraniloyl, and the like.

In the context of the present invention, the term “cleaving agent” refers to any chemical, biological or physical agent which is able to remove (or cleave) a reversible 3′-blocking group from a reversible terminating nucleoside triphosphate.

In one embodiment, the cleaving agent is a chemical cleaving agent. In one embodiment, the cleaving agent is an enzymatic cleaving agent. In one embodiment, the cleaving agent is a physical cleaving agent.

It will be understood by the one skilled in the art that the selection of a cleaving agent is dependent on the type of 3′-blocking group used. For instance, tris(2-carboxyethyl)phosphine (TCEP) can be used to cleave a 3′-O-azidomethyl group, palladium complexes can be used to cleave a 3′-O-allyl group, sodium nitrite can be used to cleave a 3′-aminoxy group, and UV light can be used to cleave a 3′-O-nitrobenzyl group.

In one embodiment, the cleaving agent may be used in conjunction with a cleavage solution comprising a denaturant (such as, e.g., urea, guanidinium chloride, formamide or betaine). In particular, adding a denaturant provides the advantage of disrupting any undesirable secondary structures in the synthetized nucleic acid. The cleavage solution may further comprise one or more buffers, which will be dependent on the exact cleavage chemistry and cleaving agent used.

In one embodiment, the 3′-blocking group is irreversible, and addition of a non-reversible terminating nucleoside triphosphate to the synthetized nucleic acid terminates the synthesis. Such irreversible 3′-blocking groups may be useful, e.g., as fluorophores, labels, tags, etc.

Example of irreversible 3′-blocking groups include, but are not limited to, fluorophores, such as, e.g., methoxycoumarin, dansyl, pyrene, Alexa Fluor 350, AMCA, Marina Blue dye, dapoxyl dye, dialkylaminocoumarin, bimane, hydroxycoumarin, Cascade Blue dye, Pacific Orange dye, Alexa Fluor 405, Cascade Yellow dye, Pacific Blue dye, PyMPO, Alexa Fluor 430, NBD, QSY 35, fluorescein, Alexa Fluor 488, Oregon Green 488, BODIPY 493/503, rhodamine green dye, BODIPY FL, 2′,7′-dichlorofluorescein, Oregon Green 514, Alexa Fluor 514, 4′,5′-dichloro-2′,7′-dimethoxyfluorescein (JOE), eosin, rhodamine 6G, BODIPY R6G, Alexa Fluor 532, BODIPY 530/550, BODIPY TMR, Alexa Fluor 555, tetramethylrhodamine (TMR), Alexa Fluor 546, BODIPY 558/568, QSY 7, QSY 9, BODIPY 564/570, lissamine rhodamine B, rhodamine red dye, BODIPY 576/589, Alexa Fluor 568, X-rhodamine, BODIPY 581/591, BODIPY TR, Alexa Fluor 594, Texas Red dye, naphthofluorescein, Alexa Fluor 610, BODIPY 630/650, malachite green, Alexa Fluor 633, Alexa Fluor 635, BODIPY 650/665, Alexa Fluor 647, QSY 21, Alexa Fluor 660, Alexa Fluor 680, Alexa Fluor 700, Alexa Fluor 750, Alexa Fluor 790, and the like.

Further examples of irreversible 3′-blocking groups include, but are not limited to, biotin or desthiobiotin groups.

In any one of the above embodiments, the nucleoside triphosphate is a 2′-protected nucleoside triphosphate.

By “2′-protected nucleoside triphosphate”, it is referred to nucleoside triphosphates which have an additional group (hereafter, “2′-protecting group”) on their 2′-end (i.e., at position 2 of their 5-carbon sugar). A particular—although not the sole—purpose of such 2′-protecting groups is to protect the reactive 2′-hydroxyl group in the specific case of ribonucleotide triphosphates.

Any 3′-blocking groups described above, whether reversible or irreversible, are also suitable to serve as 2′-protecting groups.

Additionally, any 3′-blocking groups described above, whether reversible or irreversible, can further be added at any position of the nucleoside triphosphates, whether on their 5-carbon sugar moiety and/or on their nitrogenous base.

In one embodiment, the method for ab-initio synthesis of nucleic acids comprises the following steps:
a) providing a nucleotide with a free 3′-hydroxyl group;
b) contacting said nucleotide with a (optionally, selected) nucleoside triphosphate (or a combination of (optionally, selected) nucleoside triphosphates) in the presence of an archaeal DNA primase or a functionally active fragment and/or variant thereof;
   thereby covalently binding said (optionally, selected) nucleoside triphosphate to the free 3′-hydroxyl group of the nucleotide.

In one embodiment, the method according to the present invention is for ab-initio synthesis of nucleic acids with a random sequence, and it comprises the following steps: a) providing a nucleotide with a free 3′-hydroxyl group;
b) contacting said nucleotide with a combination of (optionally, selected) nucleoside triphosphates in the presence of an archaeal DNA primase or a functionally active fragment and/or variant thereof;
   thereby randomly covalently binding said combination of (optionally, selected) nucleoside triphosphates to the free 3′-hydroxyl group of the nucleotide.

In one embodiment, the method according to the present invention is for ab-initio, sequence-controlled synthesis of nucleic acids, and it comprises the following steps:
a) providing a nucleotide with a free 3′-hydroxyl group;
b) contacting said nucleotide with a selected reversibly terminating nucleoside triphosphate in the presence of an archaeal DNA primase or a functionally active fragment and/or variant thereof,
   thereby covalently binding said selected reversibly terminating nucleoside triphosphate to the free 3′-hydroxyl group of the nucleotide;
c) applying a washing solution to remove all reagents, in particular to remove unbound reversibly terminating nucleoside triphosphates;
d) cleaving the reversible 3′-blocking group of the covalently bound terminating nucleoside triphosphate in the presence of a cleaving agent;
   thereby obtaining a nucleotide with a free 3′-hydroxyl group;
e) optionally, applying a washing solution to remove all reagents, in particular to remove the cleaving agent;
f) optionally, reiterating steps b) to e) multiple times to synthetize a nucleic acid of desired length and nucleotide sequence.

In one embodiment, more than 1 nucleoside triphosphate is added to the nucleotide with a free 3′-hydroxyl group, such as, more than 2, 5, 10, 20, 30, 40, 50, 60, 70, 80, 90, 100, 200, 300, 400, 500, 600, 700, 800, 900, 1000, 1250, 1500, 1750, 2000, 2250, 2500, 2750, 3000, 4000, 5000, 6000, 7000, 8000, 9000, 10000 or even more nucleoside triphosphates are added to the nucleotide with a free 3′-hydroxyl group by reiterating steps b) to e) as many times.

In one embodiment, the method for ab-initio synthesis of nucleic acids according to the present invention is carried out in the presence of one or more buffers (e.g., Tris or cacodylate) and/or one or more salts (e.g., Na⁺, K⁺, Mg²⁺, Mn²⁺, Cu²⁺, Zn²⁺, Co²⁺, etc., all with appropriate counterions, such as Cl⁻).

In one embodiment, the method for ab-initio synthesis of nucleic acids according to the present invention is carried out in the presence of one or more divalent cations (e.g., Mg²⁺, Mn²⁺, Co²⁺, etc., all with appropriate counterions, such as Cl⁻), preferably in the presence of M²⁺.

In one embodiment, the method for ab-initio synthesis of nucleic acids according to the present invention is carried out at a temperature ranging from about from about 60° C. to about 95° C. In one embodiment, the method for ab-initio synthesis of nucleic acids according to the present invention is carried out at a temperature of about 60° C., 65° C., 70° C., 75° C., 80° C., 85° C., 90° C. or 95° C.

In one embodiment, the method for ab-initio synthesis of nucleic acids according to the present invention is carried out in absence of eukaryotic enzyme, in particular in absence of eukaryotic polymerase (including DNA polymerase alpha and DNA polymerase beta).

In one embodiment, the archaeal DNA primase or functionally active fragment and/or variant thereof may be used in the method for ab-initio single-stranded nucleic acid synthesis according to the present invention, for cleaning-up contaminating nucleoside triphosphates comprising a free 3′-hydroxyl group in a pool of terminating nucleoside triphosphates. Indeed, commercially available pools of terminating nucleoside triphosphates typically comprise a few percent of “non-terminating” nucleoside triphosphates (i.e., comprising a free 3′-hydroxyl group) which can cause deleterious effects during the synthesis of nucleic acids.

In one embodiment, the archaeal DNA primase or functionally active fragment and/or variant thereof may be used in the method for ab-initio synthesis of nucleic acids according to the present invention, for producing synthetic homo- and heteropolymers. One skilled in the art is familiar with means and methods for producing synthetic homo- and heteropolymers, described in, e.g., Bollum, 1974 (In Boyer [Ed.], The enzymes [3^{rd} ed., Vol. 10, pp. 145-171]. New York, N.Y.: Academic Press), the content of which is incorporated herein by reference.

In one embodiment, the archaeal DNA primase or functionally active fragment and/or variant thereof may be used in the method for ab-initio synthesis of nucleic acids according to the present invention, for homopolymeric tailing of any type of 3′-OH terminus. One skilled in the art is familiar with means and methods for homopolymeric tailing, described in, e.g., Deng & Wu, 1983 (Methods Enzymol. 100:96-116) and Eschenfeldt et al., 1987 (Methods Enzymol. 152:337-342), the content of which is incorporated herein by reference.

In one embodiment, the archaeal DNA primase or functionally active fragment and/or variant thereof may be used in the method for ab-initio synthesis of nucleic acids according to the present invention, for oligonucleotide, DNA, and RNA labeling. One skilled in the art is familiar with means and methods for labelling, described in, e.g., Deng & Wu, 1983 (Methods Enzymol. 100:96-116), Tu & Cohen, 1980 (Gene. 10(2):177-183), Vincent et al., 1982 (Nucleic Acids Res. 10(21):6787-6796), Kumar et al., 1988 (Anal Biochem. 169(2):376-382), Gaastra & Klemm, 1984 (In Walker et al. [Eds.], Nucleic acids [Vol. 2, Methods in molecular biology, pp. 269-271]. Clifton, N.J.: Humana Press), Igloi & Schiefermayr, 1993 (Biotechniques. 15(3):486-497) and Winz et al., 2015 (Nucleic Acids Res. 43(17):e110), the content of which is incorporated herein by reference.

In one embodiment, the archaeal DNA primase or functionally active fragment and/or variant thereof may be used in the method for ab-initio synthesis of nucleic acids according to the present invention, for 5′-RACE (Rapid Amplification of cDNA Ends). One skilled in the art is familiar with means and methods for 5′-RACE, described in, e.g., Scotto-Lavino et al., 2006 (Nat Protoc. 1(6):2555-62), the content of which is incorporated herein by reference.

In one embodiment, the archaeal DNA primase or functionally active fragment and/or variant thereof may be used in the method for ab-initio synthesis of nucleic acids according to the present invention, for in situ localization of apoptosis, such as TUNEL (terminal deoxynucleotidyl transferase dUTP nick end labeling) assay. One skilled in the art is familiar with means and methods for in situ localization of apoptosis such as TUNEL assay, described in, e.g., Gorczyca et al., 1993 (Cancer Res. 53(8):1945-1951) and Lebon et al., 2015 (Anal Biochem. 480:37-41), the content of which is incorporated herein by reference.

In a third aspect, the present invention relates to a system for ab-initio synthesis of nucleic acids, comprising:
a nucleotide with a free 3′-hydroxyl group, optionally wherein said nucleotide is immobilized onto a support;
nucleoside triphosphates; and
an archaeal DNA primase or a functionally active fragment and/or variant thereof.

In one embodiment, the system is suitable for template-independent synthesis of nucleic acids with a random sequence, and it comprises:
a nucleotide with a free 3′-hydroxyl group, optionally wherein said nucleotide is immobilized onto a support;
a combination of (optionally, selected) nucleoside triphosphates, wherein said (optionally, selected) nucleoside triphosphates are not terminating nucleoside triphosphates; and
an archaeal DNA primase or a functionally active fragment and/or variant thereof.

In one embodiment, the system is suitable for template-independent, sequence-controlled synthesis of nucleic acids, and it comprises:
a nucleotide with a free 3′-hydroxyl group, optionally wherein said nucleotide is immobilized onto a support;
reversibly terminating selected nucleoside triphosphates, wherein different nucleoside triphosphates are not combined together in a same vial;
a cleaving agent; and
an archaeal DNA primase or a functionally active fragment and/or variant thereof.

In a fourth aspect, the present invention relates to a kit comprising:
a nucleotide with a free 3′-hydroxyl group, optionally wherein said nucleotide is immobilized onto a support;
nucleoside triphosphates; and
an archaeal DNA primase or a functionally active fragment and/or variant thereof.

In one embodiment, the kit comprises:
a nucleotide with a free 3′-hydroxyl group, optionally wherein said nucleotide is immobilized onto a support;
a combination of (optionally, selected) nucleoside triphosphates, wherein said (optionally, selected) nucleoside triphosphates are not terminating nucleoside triphosphates; and
an archaeal DNA primase or a functionally active fragment and/or variant thereof.

In one embodiment, the kit comprises:
a nucleotide with a free 3′-hydroxyl group, optionally wherein said nucleotide is immobilized onto a support;
reversibly terminating selected nucleoside triphosphates, wherein different nucleoside triphosphates are not combined together in a same vial;
a cleaving agent; and
an archaeal DNA primase or a functionally active fragment and/or variant thereof.

### BRIEF DESCRIPTION OF THE DRAWINGS

FIG. 1 is a photograph of an electrophoresis gel (SDS-PAGE) showing the purification of respectively PolpP12_{Δ297-898}, PolpTN2_{Δ311-923}, PolpTCIR10_{Δ303-928}, PolpTpep_{Δ295-914} and PolpTcele_{Δ295-913}. MW ladder: molecular weight ladder (left lane).

FIG. 2 is a photograph of a 15% urea-PAGE showing a template-independent nucleic acid synthesis assay using PolpTN2_{Δ311-923} [PolpTN2_{Δ}] or PolpP12_{Δ297-898} [PolpP12_{Δ}], at 60° C., 70° C. or 80° C. [a]: initiator sequence only, no enzyme, no dNTP; [b]: initiator sequence+enzyme, no dNTP; [c]: initiator sequence+enzyme+dNTP mix (unprotected).

FIGS. 3A-C are a set of three photographs of a 1.5% agarose gel electrophoresis showing a template-independent nucleic acid synthesis assay using PolpP12_{Δ297-898} or PolpTN2_{Δ311-923} at 70° C., 80° C., 90° C. and 100° C., in comparison with a negative control performed at 70° C. without enzyme [No Enzyme]. dsDNA LF Ladder: SmartLadder 200 to 10000 bp (Eurogentec).
FIG. 3A: red channel, Cy5 fluorescence at 675 nm;
FIG. 3B: green channel, Sybr green II fluorescence at 520 nm;
FIG. 3C: merge of red and green channels.

FIGS. 4A-C are a set of three photographs of a 1.5% agarose gel electrophoresis showing a template-independent nucleic acid synthesis assay using PolpP12_{Δ297-898} or PolpTN2_{Δ311-923} and carried out in the presence or in the absence of dNTPs and/or the initiator sequence (bearing the Cy5 fluorophore in 5′). Reactions were performed at 80° C. in the presence or in the absence of each substrate (lanes 1 to 8). Lane 9 shows a two steps reaction in which dNTPs were first incubated with either PolpP12_{Δ297-898} or PolpTN2_{Δ311-923}, during 15 minutes, followed by the addition of the initiator sequence. Left lane shows the MW ladder (SmartLadder 200 to 10000 bp (Eurogentec)).
FIG. 4A: merge of red and green channels;
FIG. 4B: green channel, Sybr green II fluorescence at 520 nm;
FIG. 4C: red channel, Cy5 fluorescence at 675 nm.

FIGS. 5A-B are a set of six photographs of a 1% agarose gel electrophoresis showing a template-independent nucleic acid synthesis assay using PolpTCIR10_{Δ303-928}, PolpTpep_{Δ295-914} or PolpTcele_{Δ295-913} and carried out at 70° C. in the presence or in the absence of a dNTPs mix or a dG/dC mix as well as in the presence or in the absence of the initiator sequence (bearing the Cy5 fluorophore in 5′). MW ladder (SmartLadder 200 to 10000 bp (Eurogentec)).
FIG. 5A: template-independent nucleic acid synthesis assay using PolpTCIR10_{Δ303-928} or PolpTpep_{Δ295-914};

FIG. 5B: template-independent nucleic acid synthesis assay using PolpTcele_{Δ295-913}.

FIG. 6 is a photograph of an electrophoresis gel (SDS-PAGE) showing the purification of PolpTN2_{Δ311-923}, PolpTN2_{Δ90-96Δ311-923}, PolpTN2_{Δ205-211Δ311-923} and PolpTN2_{Δ248-254Δ311-923}. PolpTN2_{Δ90-96Δ311-923} and PolpTN2_{Δ205-211Δ311-923} are in duplicate. MW ladder: molecular weight ladder.

FIGS. 7A-C are a set of three photographs of a 1.5% agarose gel electrophoresis showing a template-independent nucleic acid synthesis assay using PolpTN2_{Δ311-923}, PolpTN2_{Δ90-96Δ311-923}, PolpTN2_{Δ205-211Δ311-923} or PolpTN2_{Δ248-254Δ311-923} and carried out in the presence or in the absence of dNTPs and/or the initiator sequence (bearing the Cy5 fluorophore in 5′). Reactions were performed at 70° C. in the presence or in the absence of each substrate. MW ladder is SmartLadder 200 to 10000 bp (Eurogentec).
FIG. 7A: merge of red and green channels;
FIG. 7B: red channel, Cy5 fluorescence at 675 nm;
FIG. 7C: green channel, MidoriGreen direct fluorescence at 520 nm.

FIG. 8 is a photograph of a 15% urea-PAGE showing the incorporation of protected nucleoside triphosphates (3′-O-amino-dATP and 3′-O-azidomethyl-dATP), using PolpP12_{Δ297-898} at 60° C.

FIGS. 9A-C are a set of three photographs showing the incorporation by PolpP12_{Δ297-898} of labeled nucleoside triphosphates with reversibly terminating aminoalkoxyl groups at 80° C.
FIG. 9A: 15% urea-PAGE showing the incorporation of 3′-O-amino dATP or 3′-O-amino dTTP at 80° C.;
FIG. 9B: Analysis report of 3′-O-amino dATP incorporation at 80° C. R_{f}: relative migration distance;
FIG. 9C: Analysis report of 3′-O-amino dTTP incorporation at 80° C. R_{f}: relative migration distance.

FIGS. 10A-C are a set of three photographs showing the incorporation by PolpP12_{Δ297-898} of nucleoside triphosphates labeled with 3′-O-azidomethylene groups at 80° C.
FIG. 10A: 15% urea-PAGE showing the incorporation of 3′-O-azidomethyl dATP or 3′-O-azidomethyl dTTP at 80° C.;
FIG. 10B: Analysis report of 3′-O-azidomethyl dATP incorporation at 80° C. R_{f}: relative migration distance;
FIG. 10C: Analysis report of 3′-O-azidomethyl dTTP incorporation at 80° C. R_{f}: relative migration distance.

FIGS. 11A-C are a set of three scheme showing a clean-up procedure of terminating nucleoside triphosphates, in presence of contaminating nucleoside triphosphates comprising a free 3′-hydroxyl group.
FIG. 11A: first step of ab-initio nucleic acid synthesis carried out in the presence of the DNA primases described herein, and using the contaminating stock of nucleoside triphosphates comprising a free 3′-hydroxyl group;
FIG. 11B: alternative first step of ab-initio nucleic acid synthesis carried out in the presence of the DNA primases described herein, and using the contaminating stock of nucleoside triphosphates comprising a free 3′-hydroxyl group. An excess amount of exogenous dideoxynucleoside triphosphates (ddNTP) is added to avoid incorporating terminating nucleoside triphosphate to the nascent nucleic acid strand. The ddNTP can be functionalized (e.g., with biotin);
FIG. 11C: second step of the process, comprising (1) filing a centrifugal filtration column with the sample obtained after the first step; and (2) spinning the centrifugal filtration column to separate the synthetized single stranded nucleic acid fragments and DNA primase, from the terminating nucleoside triphosphates (3′-blocked nucleoside triphosphates) and buffer.

### EXAMPLES

The present invention is further illustrated by the following examples.

### Example 1

### PolpTN2_{Δ311-923}, PolpTCIR10_{Δ303-928}, PolpTpep_{Δ295-914} and PolpTcele_{Δ295-913} have an Ab-Initio Single-Stranded Nucleic Acid Synthesis Activity

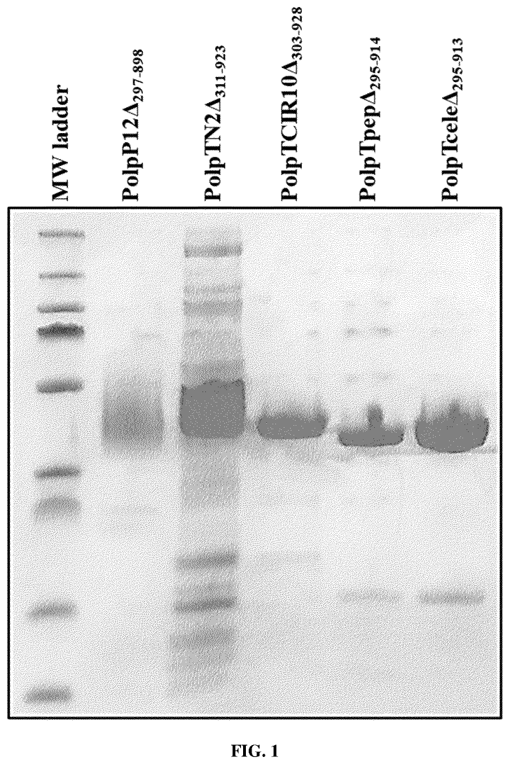

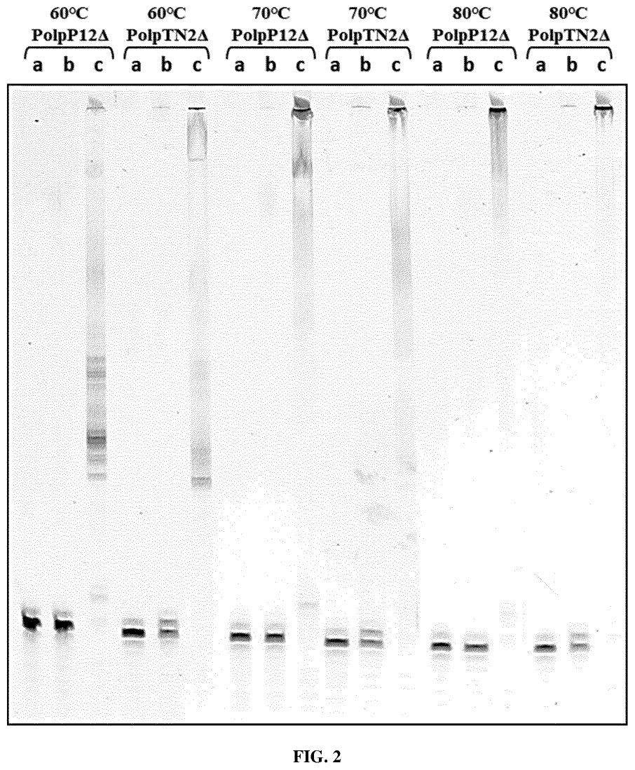

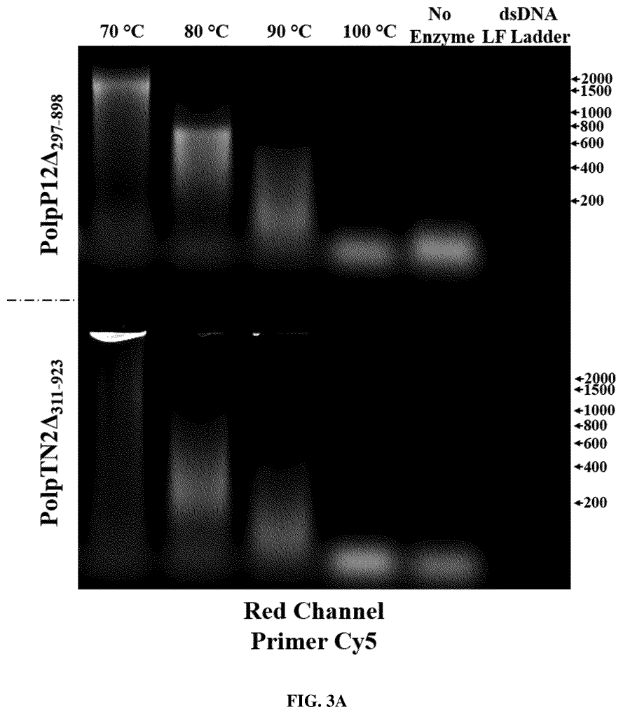

The N-terminal domain of the DNA primase from Pyrococcus sp. 12-1 (PolpP12_{Δ297-898} having the amino acid sequence of SEQ ID NO: 21), from Thermococcus nautili sp. 30-1 (PolpTN2_{Δ311-923} having the amino acid sequence of SEQ ID NO: 2), from Thermococcus sp. CIR10 (PolpTCIR10_{Δ303-928} having the amino acid sequence of SEQ ID NO: 15), from Thermococcus peptonophilus (PolpTpep_{Δ295-914} having the amino acid sequence of SEQ ID NO: 18) and from Thermococcus celericrescens (PolpTcele_{Δ295-913} having the amino acid sequence of SEQ ID NO: 20) were expressed and purified following a protocol adapted from WO2011098588 and Gill et al., 2014 (Nucleic Acids Res. 42(6):3707-3719) (FIG. 1).

SEQ ID NO: 21
MRPSDIIIDVYKAIQDHPGAGKLAIELRFYPRPTSEWIIVADIEDKAEE
LHKVLFKNNVLGKKEAYISMALHDFEEVGKKLEKLRELEEERAQKEGRK
PREVTLRNVQGEATGKVHKTVSKYTLTLVVDIDVEEIHKSKVVESEEKA
FELAKRAWDELKPKLEGIGVKPRYVFFTGGGVQLWFVAPGLEPIEVIDR
ASRVIPPVLNAMLPEGYSVDNIFDRARIVRVPLTINYKYKTPDERPLEI
RGRLIEFNDVRTPLGEVLDKLEAYAKEHGISLVTPSQARFIGTVGRYEV
DK

A template-independent nucleic acid synthesis assay was carried out with either PolpTN2_{Δ311-923} or PolpP12_{Δ297-898}, at 60° C., 70° C. and 80° C., using a single stranded nucleic acid primer as initiator sequence (bearing a Cy5 fluorophore in 5′).

Three different conditions were tested:
a: initiator sequence only; no enzyme, no dNTP;
b: initiator sequence+enzyme; no dNTP;
c: initiator sequence+enzyme+dNTP mix (unprotected).

As seen on FIG. 2, both PolpTN2_{Δ311-923} and PolpP12_{Δ297-898} exhibit an untemplated terminal nucleotidyl transferase activity for each tested temperature, when using a mixture of all four dNTPs as substrate. However, it is worth noting that at 70° C. and 80° C., most of the newly synthetized nucleic acids are several hundred bases long, hence cannot be resolved on a 15% urea-PAGE gel and remain in the well.

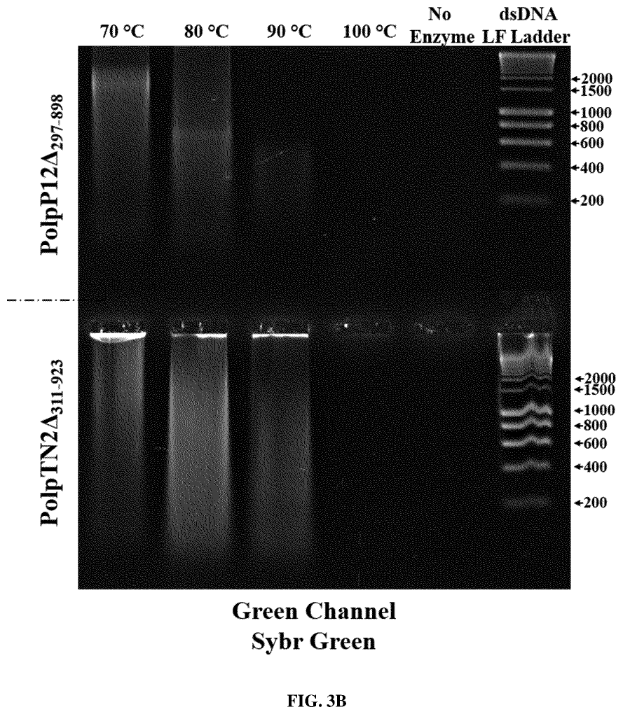

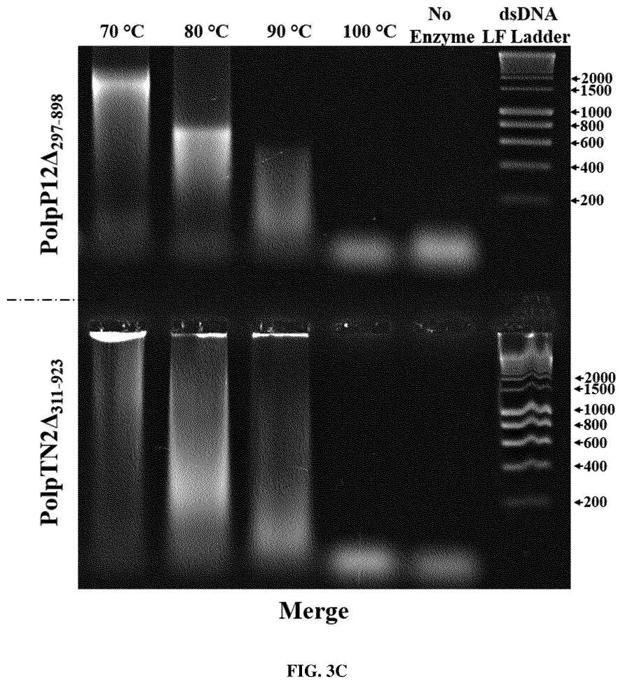

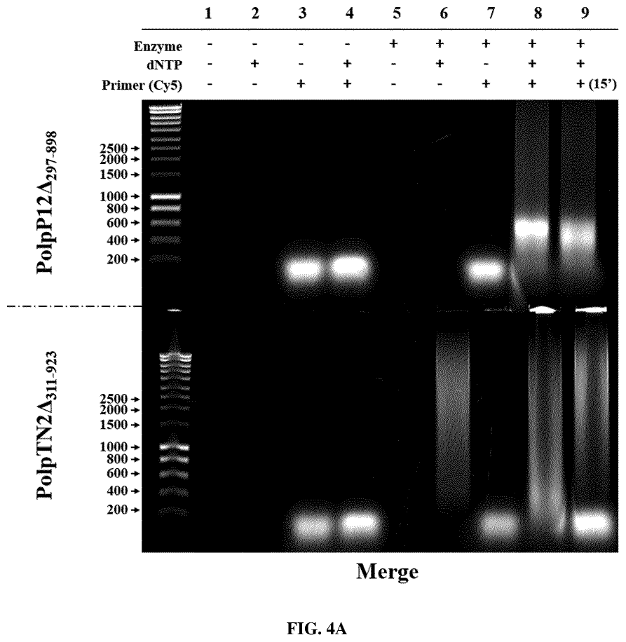

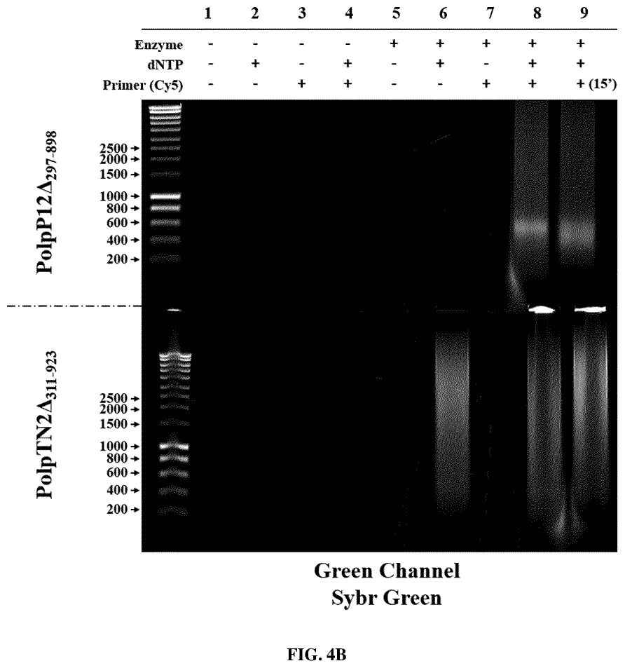

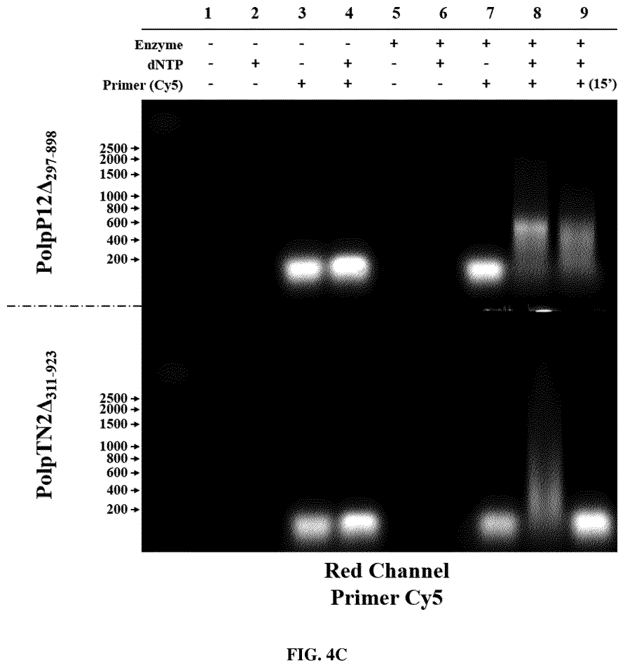

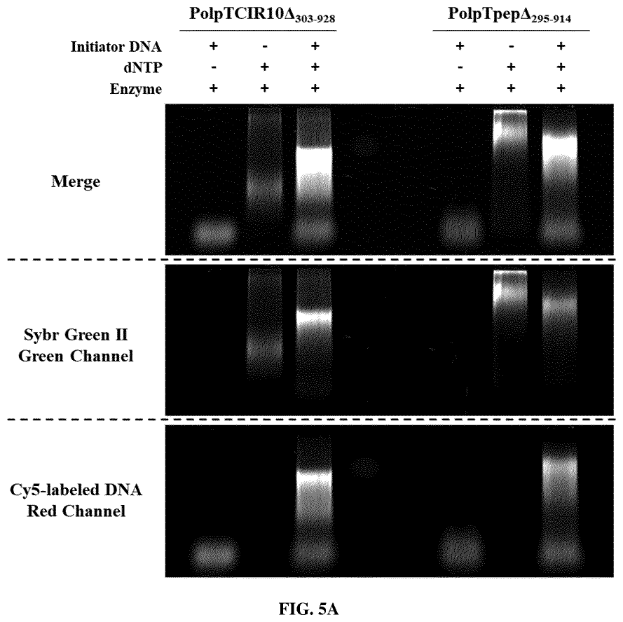

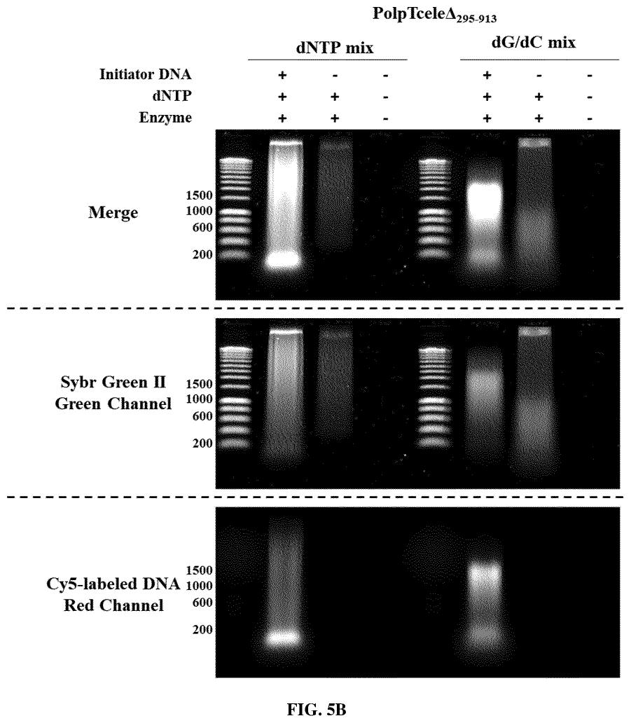

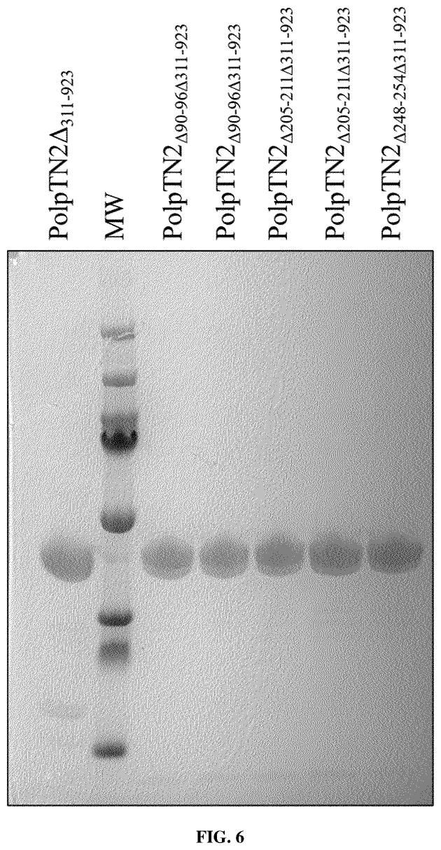

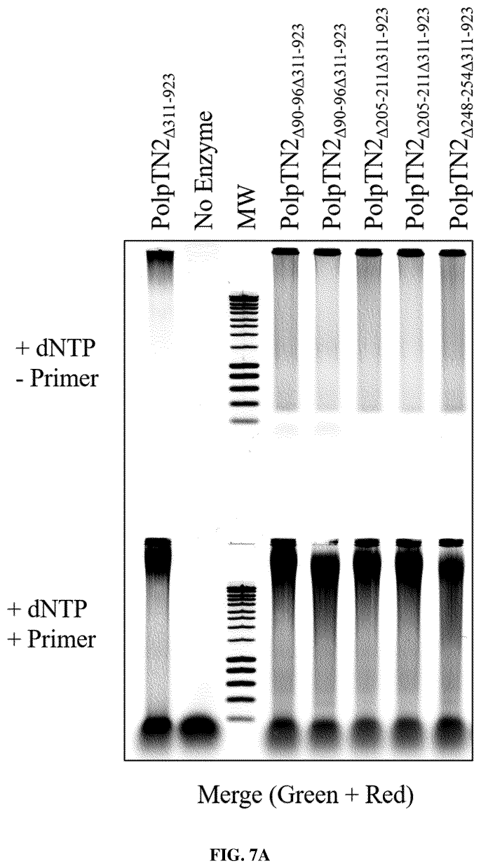

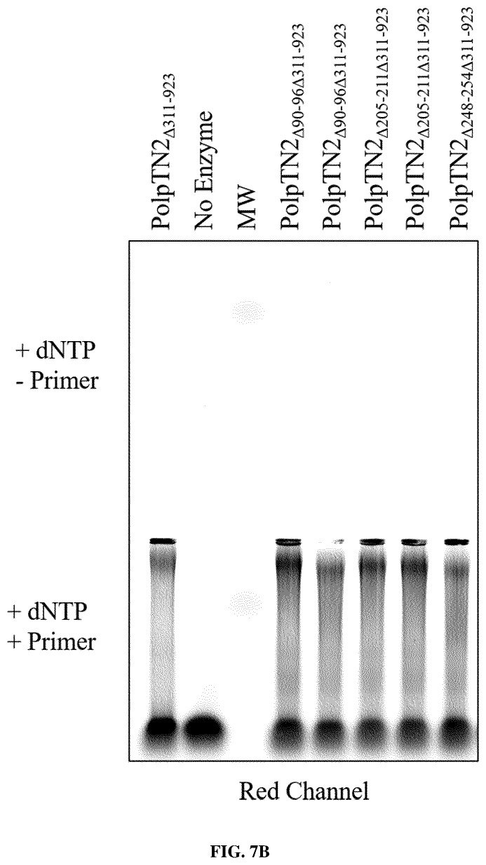

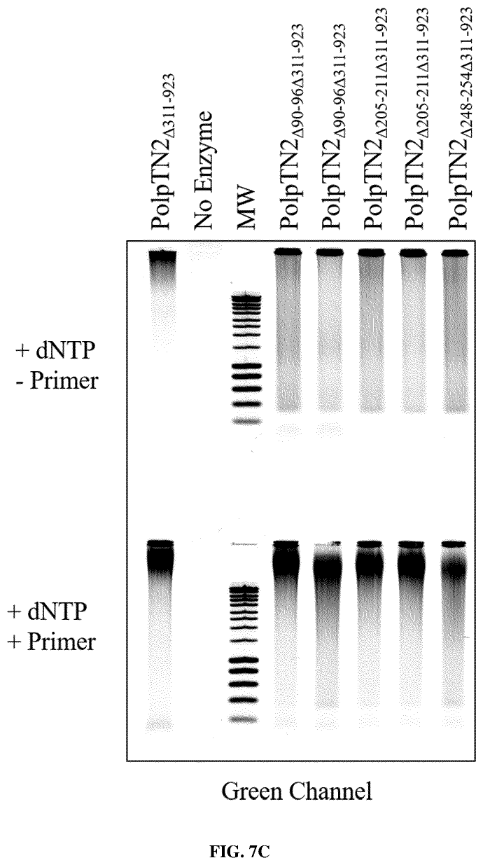

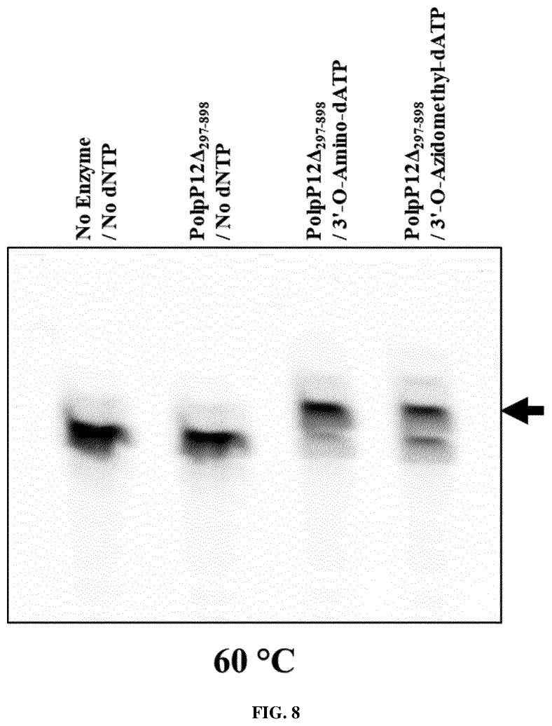

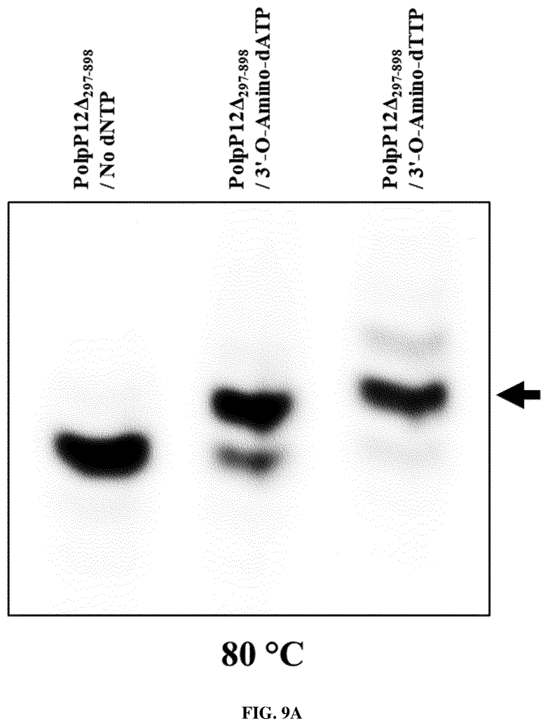

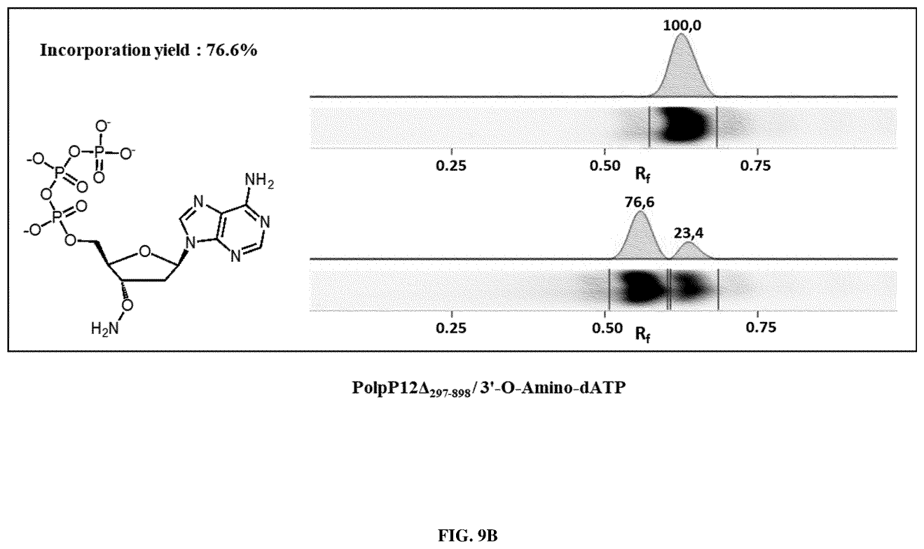

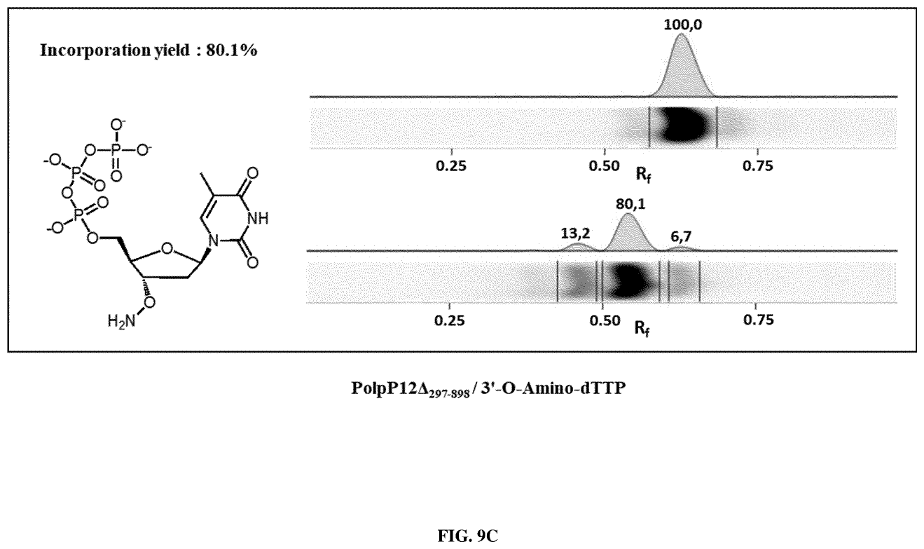

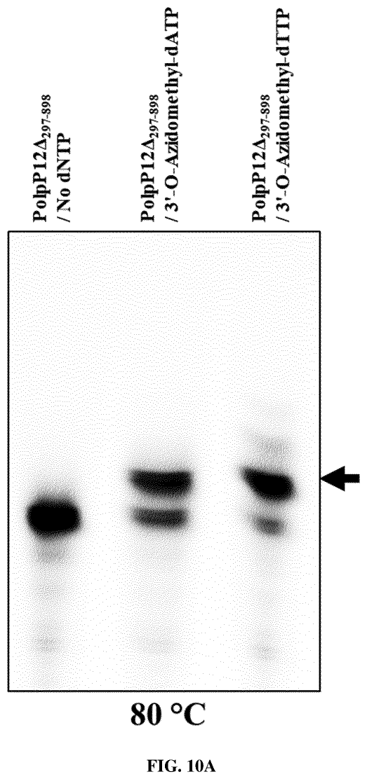

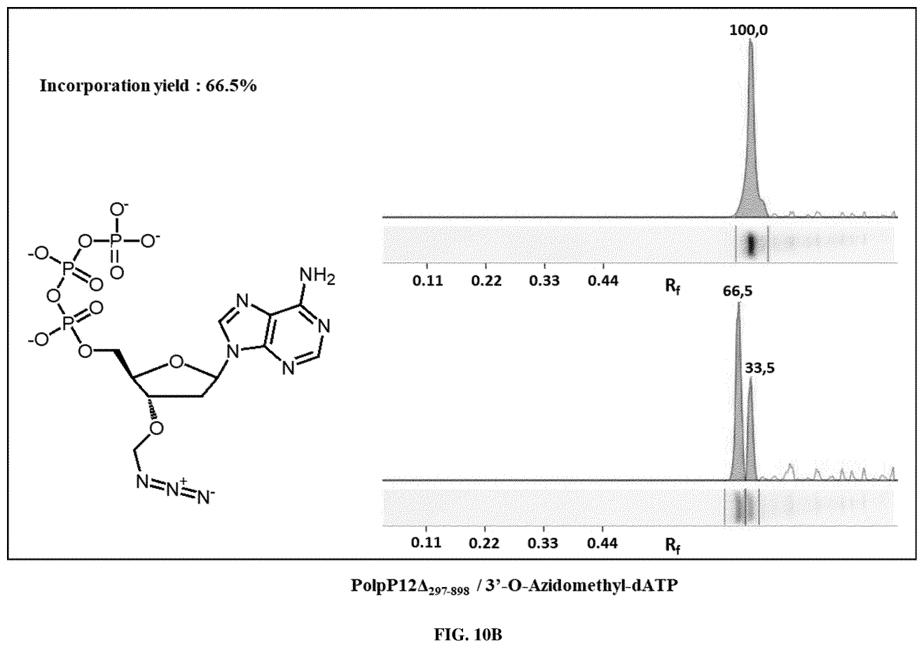

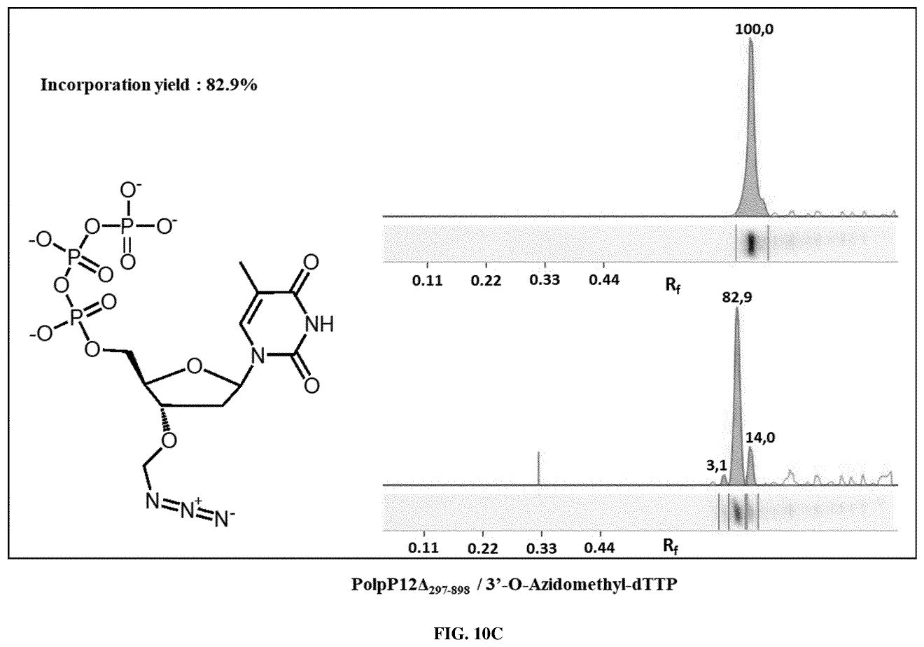

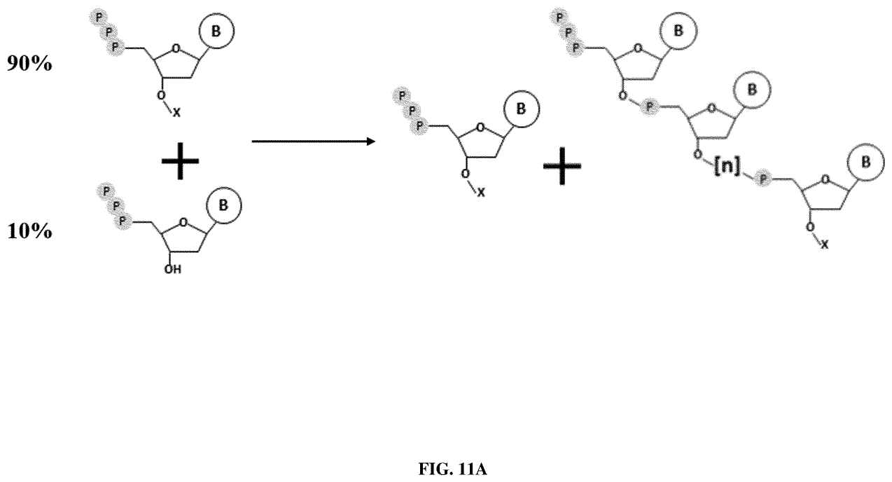

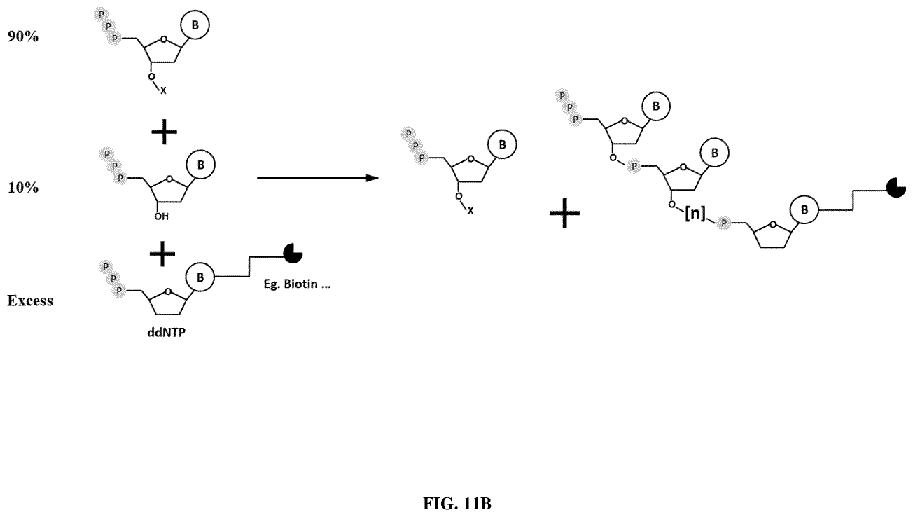

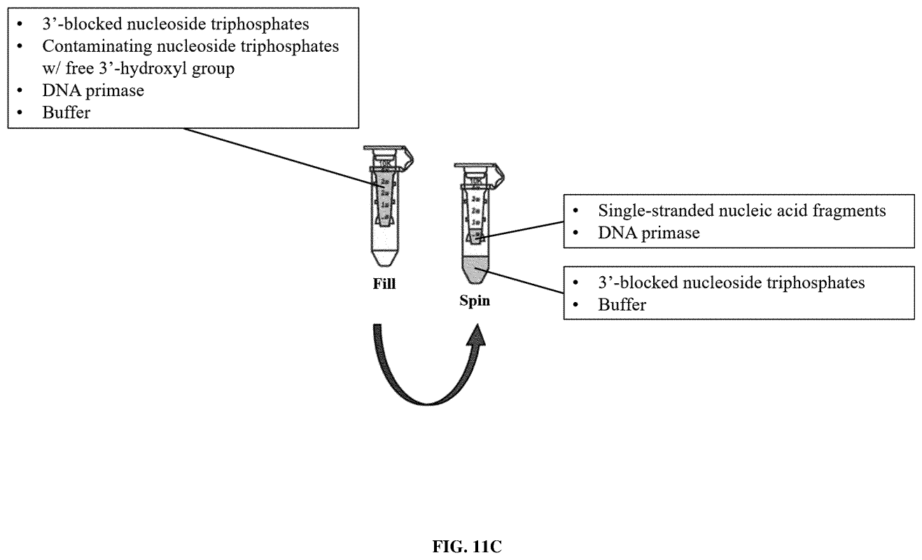

Thus, to analyze the effect of high temperatures on PolpTN2_{Δ311-923} and PolpP12_{Δ297-898} activities, a template-independent nucleic acid synthesis assay was performed as previously described, at 70° C., 80° C., 90° C. or 100° C. and resolved by agarose gel electrophoresis (FIG. 3). The terminal transferase activity was specifically evaluated by following the polymerization of the fluorescent primer (bearing a Cy5 fluorophore in 5′), recorded at 675 nm (red channel). Total nucleic acid synthesis and molecular weight markers were stained using Sybr Green II and recorded at 520 nm (green channel).

As shown on FIGS. 3A and C, both enzymes exhibit a strong template-independent terminal nucleotidyl transferase activity, which is demonstrated by the polymerization of the Cy5-labeled initiator sequence (FIG. 3A). These activities reach a maximum of polymerization at 70° C. and gradually decrease upon increasing temperatures, up to 100° C. However, in contrast to PolpP12_{Δ297-898}, PolpTN2_{Δ311-923} exhibits a diffuse migration pattern at 70° C., 80° C. and 90° C., when stained with Sybr Green II (FIG. 3B), which does not colocalize with the Cy5-labeled initiator sequence (Cf. FIGS. 3A and C). Although this intriguing result may arise from a migration issue, another explanation would be the presence of an unexpected competing activity, such as an ab-initio single-stranded nucleic acid synthesis activity.

Interestingly, Béguin et al. have demonstrated that a combination of the full length PolpTN2 primase and the PolB DNA polymerase in presence of deoxynucleotide triphosphates leads to the ab-initio synthesis of long double stranded DNA fragments (i.e., without template DNA nor oligonucleotide primer). However, this phenomenon requires the presence of both enzymes and is not observed when only PolpTN2 is reacted with a dNTP mix (Béguin et al., 2015. Extremophiles. 19(1):69-76). In contrast, our results suggest that PolpTN2_{Δ311-923} alone might be able to synthesis long fragments of single stranded nucleic acids de novo, i.e., corresponding to an ab-initio activity.

To further investigate this phenomenon, both PolpTN2_{Δ311-923} and PolpP12_{Δ297-898} were subjected to a template-independent nucleic acid synthesis assay (FIG. 4), carried out in the presence or in the absence of dNTPs and/or the initiator sequence (bearing the Cy5 fluorophore in 5′). The terminal transferase activity was specifically evaluated by following the polymerization of the fluorescent primer, recorded at 675 nm (red channel). Total nucleic acid synthesis and molecular weight markers were stained using Sybr Green II and recorded at 520 nm (green channel).

Nine different conditions were tested:
1: no enzyme, no initiator sequence, no dNTP;
2: dNTP only; no enzyme, no initiator sequence;
3: initiator sequence only; no enzyme, no dNTP;
4: initiator sequence+dNTP mix; no enzyme;
5: enzyme only, no initiator sequence, no dNTP;
6: enzyme+dNTP mix; no initiator sequence;
7: enzyme+initiator sequence; no dNTP;
8: enzyme+dNTP mix+initiator sequence;
9: enzyme+dNTP mix+initiator sequence (added after 15 minutes incubation);

As shown on FIG. 4, neither PolpP12_{Δ297-898} nor PolpTN2_{Δ311-923} is able to synthesize nucleic acids in the absence of dNTPs (FIG. 4; lanes 1, 3, 5 and 7) while the association of dNTPs with the initiator sequence leads to the synthesis of long nucleic acids fragments (FIG. 4A; lanes 8 and 9).

Interestingly, in the absence of the initiator sequence (FIG. 4; lanes 1, 2, 5 and 6) PolpTN2_{Δ311-923} readily exhibits a strong polymerase activity, upon the addition of dNTPs (FIGS. 4A and 4B; lane 6). Separated channel analysis reveals that this activity is independent of an initiator sequence, as demonstrated by the absence of Cy5 fluorescence (FIG. 4C; lane 6), thus confirming the ab-initio single-stranded nucleic acid synthesis activity of PolpTN2_{Δ311-923}.

Conversely, in the same experimental conditions, PolpP12_{Δ297-898} does not synthesize nucleic acids, as demonstrated by a total absence of fluorescence in both channels (FIGS. 4A, 4B and 4C; lane 6), indicating that this enzyme is devoid of an ab-initio single-stranded nucleic acid synthesis activity.

To further investigate the impact of such ab-initio single-stranded nucleic acid synthesis activity on the ability of PolpTN2_{Δ311-923} and PolpP12_{Δ297-898} to extend a single stranded nucleic acid fragment, a competition assay was conducted by separating both reactions (FIG. 4, lane 9). To realize this experiment, both enzymes were first incubated with dNTPs for 15 minutes, to perform a template-independent primase reaction, before adding the initiator sequence for another 15 minutes of incubation, to perform a template-independent primer extension reaction. As expected, a strong diffuse migration pattern could be observed for PolpTN2_{Δ311-923}, after Sybr Green II staining (FIG. 4B, lane 9), while the initiator sequence was found to migrate up to the dye front (FIG. 4C, lane 9), similarly to the negative control (FIG. 4C, lane 3). In contrast, PolpP12_{Δ297-898} was found to extend the initiator sequence (FIG. 4C, lane 9) as previously described, indicating that a pre-incubation with a dNTP mix does not affect its terminal nucleotidyl transferase activity. Thus, this result indicates that PolpTN2_{ΔA311-923} is not able to extent the initiator sequence in these experimental conditions.

We subsequently investigated the ability of PolpTCIR10_{Δ303-928}, PolpTpep_{Δ295-914} and PolpTcele_{Δ295-913} to perform a template-independent DNA synthesis reaction in the presence or in the absence of the initiator sequence (bearing the Cy5 fluorophore in 5′). For that purpose, PolpTCIR10_{Δ303-928} and PolpTpep_{Δ295-914} (FIG. 5A) as well as PolpTcele_{Δ295-913} (FIG. 5B) were incubated at 70° C. with or without the initiator sequence and their terminal transferase activity was evaluated by following the polymerization of the fluorescent primer, recorded at 675 nm (red channel) while their ab-initio single-stranded nucleic acid synthesis activity was evaluated through Sybr Green II staining and recorded at 520 nm (green channel).

As seen on FIGS. 5A and B, both PolpTCIR10_{Δ303-928} and PolpTpep_{Δ295-914} as well as PolpTcele_{Δ295-913} demonstrate an ability to synthesis nucleic acids ab-initio and to extend single-stranded DNA fragments, similarly to PolpTN2_{Δ311-923}. Furthermore, these activities were strengthened by the ability of PolpTcele_{Δ295-913} to catalyze both the terminal nucleotidyl transferase reaction and the ab-initio single-stranded nucleic acid synthesis reaction in the presence of a dC/dG mix (FIG. 5B), indicating that the use of these enzymes is relevant for processes requiring either an ab-initio single-stranded nucleic acid synthesis activity or a terminal nucleotidyl transferase activity.

### Example 2

### PolpTN2_{Δ311-923} Variants with Internal Deletions are Still Functional

Although PolpTN2_{Δ311-923}, PolpTCIR10_{Δ303-928}, PolpTpep_{Δ295-914} and PolpThele_{Δ295-913} present similar activities, it is worth noting that these enzymes are diverging both in term of sequence identity and length. Indeed, protein sequence alignment of these enzymes showed the presence of several loops that we suspected might be dispensable for both terminal nucleotidyl transferase and ab-initio activities in PolpTN2_{Δ311-923}. These loops are located between amino acid residues 90 to 96, 205 to 211 and 248 to 254 of PolpTN2_{Δ311-923} (reference to SEQ ID NO: 2 numbering). One similar loop was also found between amino acid residues 93 to 98 of PolpTCIR10_{Δ303-928} (reference to SEQ ID NO: 15 numbering).

This study was driven by the necessity of providing enzymes that are suitable for industrial applications, and adapted for both upstream and downstream processes. In that respect, the removal of these loops can improve on the one hand protein stability and protein expression yield as it maximizes the presence of structured regions. On the other hand, loop deletion leads to a reduced protein size, which eventually facilitates the removal of the enzyme along with other reagents by ultrafiltration during downstream purification.

To investigate the effect of loop deletion and size reduction on terminal nucleotidyl transferase and ab-initio activities, we generated variants of PolpTN2_{Δ311-923}, which presents the largest size with 310 amino acid residues versus 295 amino acid residues for PolpTpep_{Δ295-914} and PolpTcele_{Δ295-913}. This led to four variants, namely PolpTN2_{Δ90-96Δ311-923} (with SEQ ID NO: 3), PolpTN2_{Δ205-211Δ311-923} (with SEQ ID NO: 4), PolpTN2_{Δ248-254Δ311-923} (with SEQ ID NO: 5), and PolpTN2_{Δ243-254Δ311-923} (with SEQ ID NO: 6). The three first ones were expressed and purified as previously described, in simplicate or duplicate (FIG. 6).

We subsequently investigated the ability of these three variants to perform a template-independent DNA synthesis reaction in the presence or in the absence of the initiator sequence (bearing the Cy5 fluorophore in 5′).

For that purpose, PolpTN2_{Δ90-96Δ311-923}, PolpTN2_{Δ205-211Δ311-923} and PolpTN2_{Δ248-254Δ311-923}, along with of PolpTN2_{Δ311-923} as control, (FIG. 7) were incubated at 70° C. with or without the initiator sequence and their terminal transferase activity was evaluated by following the polymerization of the fluorescent primer, recorded at 675 nm (red channel) (FIG. 7B) while their ab-initio single-stranded nucleic acid synthesis activity was evaluated through MidoriGreen direct staining and recorded at 520 nm (green channel) (FIG. 7C).

As seen on FIGS. 7A to C, all tested variants demonstrated an ability to synthesis nucleic acids ab-initio and to extend single-stranded DNA fragments, similarly to PolpTN2_{Δ311-923} and as previously demonstrated for PolpTCIR10_{Δ303-928}, PolpTpep_{Δ295-914} and PolpTcele_{Δ295-913.}

These results hence demonstrate the possibility of shaping these enzymes to optimally integrate them into industrial processes that require downstream steps, such as ultrafiltration.

Moreover, since each of the three loop deletions, taken individually, is not detrimental to the activity of the enzyme, it is expectable that:
combinations of two or even three loop deletions in the same PolpTN2_{Δ311-923} construct would also lead to a functional enzyme (SEQ ID NOs: 7 to 13); and
deletion of the corresponding loop in PolpTCIR10_{Δ303-928} would also lead to a functional enzyme (SEQ ID NO: 16).

### Example 3

### Commercially Available Protected Nucleoside Triphosphate Pools are not Devoid of Impurities

A terminal transferase activity assay was carried out with PolpP12_{Δ297-898} at 60° C. (FIG. 8) using 3′-O-amino dATP or 3′-O-azidomethyl dATP and a single-stranded nucleic acid primer as initiator sequence (bearing a Cy5 fluorophore in 5′).

Four different conditions were tested:
a: initiator sequence only; no enzyme, no dNTP at 60° C.;
b: initiator sequence+enzyme; no dNTP at 60° C.;
c: initiator sequence+enzyme+3′-O-amino dATP at 60° C.;
d: initiator sequence+enzyme+3′-O-azidomethyl dATP at 60° C.;

Thus, PolpP12_{Δ297-898} was found to naturally incorporate 3′-reversible terminating nucleotides at 60° C., as demonstrated by the higher migration pattern of the initiator primer, when compared to the negative control (FIG. 8).

To further investigate the effect of higher temperatures on the ability of PolpP12_{Δ297-898} to incorporate 3′-reversible terminating nucleotides, a terminal transferase activity assay was carried out 80° C. using 3′-O-amino dNTPs (FIG. 9) or 3′-O-azidomethyl dNTP (FIG. 10) and a single-stranded nucleic acid primer as initiator sequence (bearing a Cy5 fluorophore in 5′).

Three different conditions were tested in each case:
initiator sequence+enzyme; no dNTP at 80° C.;
initiator sequence+enzyme+3′-O-amino-dATP or 3′-O-azidomethyl dATP at 80° C.;
initiator sequence+enzyme+3′-O-amino dTTP or 3′-O-azidomethyl dTTP at 80° C.

As previously shown, PolpP12_{Δ297-898} was found to efficiently incorporate 3′-reversible terminating nucleotides at 80° C., as demonstrated by the higher migration pattern of the initiator primer, when compared to negative controls (FIGS. 9A and 10A).

Furthermore, it was found to incorporate both purine-type and pyrimidine-type nucleobases, with a yield of 76.6% and 80.1% for 3′-O-amino dATP and 3′-O-amino dTTP respectively (FIGS. 9B and C), while the incorporation 3′-O-azidomethyl dATP and 3′-O-azidomethyl dTTP led to yields of 66.5% and 82.9%, respectively (FIGS. 10B and C).

Despite these performances, FIGS. 8, 9A and 10A evidence that a polyaddition of 2 to 3 nucleoside triphosphates seems to occur at different levels.

In addition, quality control reports provided by the oxime-blocked nucleoside triphosphates' manufacturers indicate a purity ratio of around 90%. This seems to be consistent with our various observations.

This small percentage of impurity has extremely detrimental effects in the controlled synthesis of nucleic acids.

### Example 4

### Clean-Up Procedure of Terminating Nucleotides Prior to their Use in Nucleic Acids Synthesis

The means and methods described herein can be used for cleaning-up of contaminating nucleoside triphosphates comprising a free 3′-hydroxyl group in a pool of terminating nucleoside triphosphates. Using these means and methods, the costs of the whole clean-up procedure are considerably reduced, since template, primer, or solid support are not needed; moreover, the scope of nucleoside triphosphates that can be purified is wide:deoxyribonucleoside, ribonucleotides, chemical synthesis intermediates, etc.

Ab-Initio Synthesis Nucleic Acid Synthesis

Each pool of 3′-blocked nucleoside triphosphates at a concentration ranging from 200 μM to 5 mM is incubated in a buffer comprising 50 mM Tris-HCl (pH 8.0), 5 mM manganese chloride (MnC12), and the functionally active fragment of the DNA primase from Thermococcus nautili sp. 30-1 (with SEQ ID NO: 2), Thermococcus sp. CIR10 (with SEQ ID NO: 15), Thermococcus peptonophilus (with SEQ ID NO: 18), or Thermococcus celericrescens (with SEQ ID NO: 20) at a concentration ranging from 5 μM to 50 μM).

The targeted concentration of the initial pool of nucleosides triphosphates is calculated to obtain at least the purified 3′-blocked nucleoside triphosphates at a final concentration of 10×, thus ready to be used for different applications such as sequence-controlled, template-independent DNA synthesis.

The mix is incubated at 70° C. for 1 hour. The enzymatic reaction is then stopped by the addition of 12.5 mM EDTA (FIG. 11A).

Optionally, exogenous dideoxynucleoside triphosphates can be added in excess, to avoid incorporating terminating nucleoside triphosphate to the nascent nucleic acid strand (FIG. 11B). Such exogenous dideoxynucleoside triphosphates can, e.g., be functionalized to be further affinity purified.

Isolation of the 3′-Blocked Nucleoside Triphosphates

In presence of contaminating nucleoside triphosphates comprising a free 3′-hydroxyl group, the enzymatic reaction generates long single stranded nucleic acid fragments ranging from about 15 to hundreds of nucleotides long.

Purification of the 3′-blocked nucleoside triphosphates can be performed using centrifugal filtration columns, such as, e.g., Amicon® Ultra 0.5 (Merck Millipore) with a molecular weight cut-off ranging from 3 to 30 kD. Such device provides the best balance between recovery and spin time for synthetized nucleic acid and enzyme retention and release of 3′-blocked nucleoside triphosphates (FIG. 11C).

Hence, at the end of this filtration step, not only the synthetized nucleic acid and enzyme are retained, but above all, the 3′-blocked nucleoside triphosphates are directly recovered in the filtrate at the right concentration (10×), and in the suitable activity buffer for the next step.

Alternatively, the same result could be obtained using a HPLC system with, e.g., an anion-exchange medium (such as MiniQ™ from Cytiva, formerly GE Healthcare), or an affinity medium (depending on the functional group borne by exogenous dideoxynucleoside triphosphates added in excess).

1. A method for ab-initio single-stranded nucleic acid synthesis, comprising contacting the free 3′-hydroxyl group of a nucleotide with at least one nucleoside triphosphate, or a combination of nucleoside triphosphates, in the presence of a primase domain of an archaeal DNA primase belonging to the primase-polymerase family or a functionally active fragment and/or variant thereof,
wherein said primase domain consists of the N terminal domain of an archaeal DNA primase from an archaeon of the Thermococcus genus,
wherein the functionally active fragment and/or variant thereof retains its capabilities of both ab initio single stranded nucleic acid synthesis activity and template-independent terminal nucleotidyl transferase activity, thereby covalently binding said nucleoside triphosphate to the free 3′-hydroxyl group of the nucleotide, and
wherein the method is carried out in absence of both complementary nucleic acid template and initiator sequence.

2. (canceled)

3. The method according to claim 1, wherein said archaeal DNA is selected from the group consisting of Thermococcus nautili sp. 30-1 DNA primase, Thermococcus sp. CIR10 DNA primase, Thermococcus peptonophilus DNA primase, and Thermococcus celericrescens DNA primase.

4. The method according to claim 1, wherein said archaeal DNA is:
Thermococcus nautili sp. 30-1 DNA primase having the amino acid sequence of SEQ ID NO: 1;
Thermococcus sp. CIR10 DNA primase having the amino acid sequence of SEQ ID NO: 14;
Thermococcus peptonophilus DNA primase having the amino acid sequence of SEQ ID NO: 17; or
Thermococcus celericrescens DNA primase having the amino acid sequence of SEQ ID NO: 19.

5. The method according to claim 1, wherein said primase domain is the primase domain of:
the Thermococcus nautili sp. 30-1 DNA primase having the amino acid sequence of any one of SEQ ID NOs: 2 to 13; or
the Thermococcus sp. CIR10 DNA primase having the amino acid sequence of any one of SEQ ID NOs: 15 or 16; or
the Thermococcus peptonophilus DNA primase having the amino acid sequence of SEQ ID NO: 18; or
the Thermococcus celericrescens DNA primase having the amino acid sequence of SEQ ID NO: 20;
or a functionally active fragment and/or variant thereof:
having at least 70% sequence identity with the amino acid sequence of any one of SEQ ID NOs: 2 to 13, 15, 16, 18 or 20; and
being capable of template-independent terminal nucleotidyl transferase activity; and
being capable of ab-initio single-stranded nucleic acid synthesis activity.

6. The method according to claim 1, wherein said primase domain of an archaeal DNA primase belonging to the primase-polymerase family is the primase domain of:
the Thermococcus nautili sp. 30-1 DNA primase having the amino acid sequence of any one of SEQ ID NOs: 2 to 5; or
the Thermococcus sp. CIR10 DNA primase having the amino acid sequence of SEQ ID NO: 15; or
the Thermococcus peptonophilus DNA primase having the amino acid sequence of SEQ ID NO: 18; or
the Thermococcus celericrescens DNA primase having the amino acid sequence of SEQ ID NO: 20;
or a functionally active fragment and/or variant thereof:
having at least 70% sequence identity with said amino acid sequence; and
being capable of ab-initio single-stranded nucleic acid synthesis activity; and
being capable of template-independent terminal nucleotidyl transferase activity.

7. The method according to claim 1, wherein the nucleotide is immobilized onto a support.

8. The method according to claim 1, wherein the ab-initio single-stranded nucleic acid synthesis is carried out at a temperature ranging from about 60° C. to about 95° C.

9. The method according to claim 1, wherein said method is for ab-initio synthesis of nucleic acids with random nucleotide sequence, and the at least one nucleoside triphosphate does not comprise terminating nucleoside triphosphates.

10. The method according to claim 1, wherein said method is for ab-initio sequence-controlled synthesis of nucleic acids, and the at least one nucleoside triphosphate is a terminating nucleoside triphosphate comprising a reversible 3′-blocking group.

11. The method according to claim 10, comprising the steps of:
a) providing the nucleotide with a free 3′-hydroxyl group;
b) contacting said nucleotide with a terminating nucleoside triphosphate in the presence of the primase domain of the archaeal DNA primase belonging to the primase-polymerase family or the functionally active fragment and/or variant thereof,
thereby covalently binding said terminating nucleoside triphosphate to the free 3′-hydroxyl group of the nucleotide;
c) applying a washing solution to remove all reagents, in particular to remove unbound terminating nucleoside triphosphates;
d) cleaving the reversible 3′-blocking group of the covalently bound terminating nucleoside triphosphate in the presence of a cleaving agent, and
thereby obtaining a nucleotide with a free 3′-hydroxyl group.

12. The method according to claim 1, wherein said method is for cleaning-up contaminating nucleoside triphosphates comprising a free 3′-hydroxyl group in a pool of terminating nucleoside triphosphates.

13. An isolated functionally active fragment of an archaeal DNA primase consisting of an amino acid sequence of any one of SEQ ID NOs: 3 to 13, 15, 16, 18 or 20, or a functionally active fragment and/or variant thereof:
having at least 70% sequence identity with said amino acid sequence;
being capable of ab-initio single-stranded nucleic acid synthesis activity; and
being capable of template-independent terminal nucleotidyl transferase activity.

14. The isolated functionally active fragment of the archaeal DNA primase or variant thereof according to claim 13, consisting of the amino acid sequence of any one of SEQ ID NOs: 3 to 13, 15, 16, 18 or 20.

15. (canceled)

16. (canceled)

17. A nucleic acid encoding the functionally active fragment of an archaeal DNA primase according to claim 13 or an expression vector comprising the nucleic acid operably linked to regulatory elements, or a host cell comprising the expression vector.

18. (canceled)

19. (canceled)

20. (canceled)

21. (canceled)

22. The method according to claim 11, further comprising the step of:
(e) applying a washing solution to remove all reagents, in particular to remove the cleaving agent;

23. The method according to claim 22, further comprising the step of:
(f) reiterating steps b) to e) multiple times to synthetize the nucleic acid until desired length and nucleotide sequence.

24. The nucleic acid according to claim 17, wherein the nucleic acid is operably linked to a promoter.


SEQUENCE LISTING (21 sequences)

SEQ ID NO: 1 - protein, 923 aa, Thermococcus nautili
  Thermococcus nautili sp. 30-1 DNA primase
MSSLRPSSIIIDIYKVIQDHPDSSRWAIELRFLPKPISSEWIFITDIEERASEIDKVLTKYNIMKKKDAYVSMAIHDFEKVTAKLKRVQEEENKKATEGERRLRRITLDKIQGDSETTSGFTLALVVDIDNTKIHDTRIIENEEEAFEASKREWEALKPKLQELGFLPRWILYTGGGLQLWFVSDKLEPISVIDRASEIIPNIMNGVNGVKGLLSEGFKADNIFDPARIVRAPLTFNHKYRTIIKDEDGTERVVPTQVKGRVIEFNDVRISLTEFLDRLEAYAKEKGIPLEKPTKRKFLELASKRYEVTSSNFEALAERLFTELRPWWEIAKEKGWSRHHLTMGIATYILRNTNLTPEQLIGSENSPGLWELVFAKLVEAGLEDPDDWKNRASTIRDAYKKIESGKKVATKAYLRKYIEGLSEEDAVQILLSVKRALLPYLKAVDVKRISKYSARPYEITEEIPKSWEDIDENRKKATGVWYIDFLALETANDIYFEDLPKPPVFYIRFVEKNKEKFKLNETLYHSFLTWLGITEGEPLDRTELIDLLVEKFGFTVEDLKAIYYRKILTLLKPEGMRTPKCIQEFLFELATEGNLPEDKIRHLAHWVKFYARPLRHSTTSVLLKGRGKPVDMRLAIWAKVVEFFAEDDEVAEELITTFKKAYMGAEPPFPCIGAESCPFYPDHRACPFIVPKRKEVLPVSIVDVQLHGSDGIVVLVGGPTEVTAFTLEGKVEWIKTTKKTVKYPIAEWFLDRFAKEYLSLPEAPSWWKLEEVTEILKSRARVVKSQFDKFEDYLEQFIEWLQKENSRRGILPYEKADENHLFIKGEWVGIPPGFAREFYSGELLIGGPTFRRMLEQKLGKDYRKMSAKIHLNTGLKDKRNCYFVSVEWFRKHVGEPNIQEITSEGDVSFDGLSYDDEEEGVVG

SEQ ID NO: 2 - protein, 310 aa, Artificial Sequence
  PolpTN2??311-923
MSSLRPSSIIIDIYKVIQDHPDSSRWAIELRFLPKPISSEWIFITDIEERASEIDKVLTKYNIMKKKDAYVSMAIHDFEKVTAKLKRVQEEENKKATEGERRLRRITLDKIQGDSETTSGFTLALVVDIDNTKIHDTRIIENEEEAFEASKREWEALKPKLQELGFLPRWILYTGGGLQLWFVSDKLEPISVIDRASEIIPNIMNGVNGVKGLLSEGFKADNIFDPARIVRAPLTFNHKYRTIIKDEDGTERVVPTQVKGRVIEFNDVRISLTEFLDRLEAYAKEKGIPLEKPTKRKFLELASKRYEVTS

SEQ ID NO: 3 - protein, 304 aa, Artificial Sequence
  PolpTN2?90-96?311-923
MSSLRPSSIIIDIYKVIQDHPDSSRWAIELRFLPKPISSEWIFITDIEERASEIDKVLTKYNIMKKKDAYVSMAIHDFEKVTAKLKRVQETEGERRLRRITLDKIQGDSETTSGFTLALVVDIDNTKIHDTRIIENEEEAFEASKREWEALKPKLQELGFLPRWILYTGGGLQLWFVSDKLEPISVIDRASEIIPNIMNGVNGVKGLLSEGFKADNIFDPARIVRAPLTFNHKYRTIIKDEDGTERVVPTQVKGRVIEFNDVRISLTEFLDRLEAYAKEKGIPLEKPTKRKFLELASKRYEVTS

SEQ ID NO: 4 - protein, 304 aa, Artificial Sequence
  PolpTN2?205-211?311-923
MSSLRPSSIIIDIYKVIQDHPDSSRWAIELRFLPKPISSEWIFITDIEERASEIDKVLTKYNIMKKKDAYVSMAIHDFEKVTAKLKRVQEEENKKATEGERRLRRITLDKIQGDSETTSGFTLALVVDIDNTKIHDTRIIENEEEAFEASKREWEALKPKLQELGFLPRWILYTGGGLQLWFVSDKLEPISVIDRASEIIPNIMNGLLSEGFKADNIFDPARIVRAPLTFNHKYRTIIKDEDGTERVVPTQVKGRVIEFNDVRISLTEFLDRLEAYAKEKGIPLEKPTKRKFLELASKRYEVTS

SEQ ID NO: 5 - protein, 304 aa, Artificial Sequence
  PolpTN2?248-254?311-923
MSSLRPSSIIIDIYKVIQDHPDSSRWAIELRFLPKPISSEWIFITDIEERASEIDKVLTKYNIMKKKDAYVSMAIHDFEKVTAKLKRVQEEENKKATEGERRLRRITLDKIQGDSETTSGFTLALVVDIDNTKIHDTRIIENEEEAFEASKREWEALKPKLQELGFLPRWILYTGGGLQLWFVSDKLEPISVIDRASEIIPNIMNGVNGVKGLLSEGFKADNIFDPARIVRAPLTFNHKYRTIIKDEDPTQVKGRVIEFNDVRISLTEFLDRLEAYAKEKGIPLEKPTKRKFLELASKRYEVTS

SEQ ID NO: 6 - protein, 298 aa, Artificial Sequence
  PolpTN2?243-254?311-923
MSSLRPSSIIIDIYKVIQDHPDSSRWAIELRFLPKPISSEWIFITDIEERASEIDKVLTKYNIMKKKDAYVSMAIHDFEKVTAKLKRVQEEENKKATEGERRLRRITLDKIQGDSETTSGFTLALVVDIDNTKIHDTRIIENEEEAFEASKREWEALKPKLQELGFLPRWILYTGGGLQLWFVSDKLEPISVIDRASEIIPNIMNGVNGVKGLLSEGFKADNIFDPARIVRAPLTFNHKYRTPTQVKGRVIEFNDVRISLTEFLDRLEAYAKEKGIPLEKPTKRKFLELASKRYEVTS

SEQ ID NO: 7 - protein, 298 aa, Artificial Sequence
  PolpTN2?90-96?205-211?311-923
MSSLRPSSIIIDIYKVIQDHPDSSRWAIELRFLPKPISSEWIFITDIEERASEIDKVLTKYNIMKKKDAYVSMAIHDFEKVTAKLKRVQETEGERRLRRITLDKIQGDSETTSGFTLALVVDIDNTKIHDTRIIENEEEAFEASKREWEALKPKLQELGFLPRWILYTGGGLQLWFVSDKLEPISVIDRASEIIPNIMNGLLSEGFKADNIFDPARIVRAPLTFNHKYRTIIKDEDGTERVVPTQVKGRVIEFNDVRISLTEFLDRLEAYAKEKGIPLEKPTKRKFLELASKRYEVTS

SEQ ID NO: 8 - protein, 298 aa, Artificial Sequence
  PolpTN2?90-96?248-254?311-923
MSSLRPSSIIIDIYKVIQDHPDSSRWAIELRFLPKPISSEWIFITDIEERASEIDKVLTKYNIMKKKDAYVSMAIHDFEKVTAKLKRVQETEGERRLRRITLDKIQGDSETTSGFTLALVVDIDNTKIHDTRIIENEEEAFEASKREWEALKPKLQELGFLPRWILYTGGGLQLWFVSDKLEPISVIDRASEIIPNIMNGVNGVKGLLSEGFKADNIFDPARIVRAPLTFNHKYRTIIKDEDPTQVKGRVIEFNDVRISLTEFLDRLEAYAKEKGIPLEKPTKRKFLELASKRYEVTS

SEQ ID NO: 9 - protein, 292 aa, Artificial Sequence
  PolpTN2?90-96?243-254?311-923
MSSLRPSSIIIDIYKVIQDHPDSSRWAIELRFLPKPISSEWIFITDIEERASEIDKVLTKYNIMKKKDAYVSMAIHDFEKVTAKLKRVQETEGERRLRRITLDKIQGDSETTSGFTLALVVDIDNTKIHDTRIIENEEEAFEASKREWEALKPKLQELGFLPRWILYTGGGLQLWFVSDKLEPISVIDRASEIIPNIMNGVNGVKGLLSEGFKADNIFDPARIVRAPLTFNHKYRTPTQVKGRVIEFNDVRISLTEFLDRLEAYAKEKGIPLEKPTKRKFLELASKRYEVTS

SEQ ID NO: 10 - protein, 298 aa, Artificial Sequence
  PolpTN2?205-211?248-254?311-923
MSSLRPSSIIIDIYKVIQDHPDSSRWAIELRFLPKPISSEWIFITDIEERASEIDKVLTKYNIMKKKDAYVSMAIHDFEKVTAKLKRVQEEENKKATEGERRLRRITLDKIQGDSETTSGFTLALVVDIDNTKIHDTRIIENEEEAFEASKREWEALKPKLQELGFLPRWILYTGGGLQLWFVSDKLEPISVIDRASEIIPNIMNGLLSEGFKADNIFDPARIVRAPLTFNHKYRTIIKDEDPTQVKGRVIEFNDVRISLTEFLDRLEAYAKEKGIPLEKPTKRKFLELASKRYEVTS

SEQ ID NO: 11 - protein, 292 aa, Artificial Sequence
  PolpTN2?205-211?243-254?311-923
MSSLRPSSIIIDIYKVIQDHPDSSRWAIELRFLPKPISSEWIFITDIEERASEIDKVLTKYNIMKKKDAYVSMAIHDFEKVTAKLKRVQEEENKKATEGERRLRRITLDKIQGDSETTSGFTLALVVDIDNTKIHDTRIIENEEEAFEASKREWEALKPKLQELGFLPRWILYTGGGLQLWFVSDKLEPISVIDRASEIIPNIMNGLLSEGFKADNIFDPARIVRAPLTFNHKYRTPTQVKGRVIEFNDVRISLTEFLDRLEAYAKEKGIPLEKPTKRKFLELASKRYEVTS

SEQ ID NO: 12 - protein, 292 aa, Artificial Sequence
  PolpTN2?90-96?205-211?248-254?311-923
MSSLRPSSIIIDIYKVIQDHPDSSRWAIELRFLPKPISSEWIFITDIEERASEIDKVLTKYNIMKKKDAYVSMAIHDFEKVTAKLKRVQETEGERRLRRITLDKIQGDSETTSGFTLALVVDIDNTKIHDTRIIENEEEAFEASKREWEALKPKLQELGFLPRWILYTGGGLQLWFVSDKLEPISVIDRASEIIPNIMNGLLSEGFKADNIFDPARIVRAPLTFNHKYRTIIKDEDPTQVKGRVIEFNDVRISLTEFLDRLEAYAKEKGIPLEKPTKRKFLELASKRYEVTS

SEQ ID NO: 13 - protein, 286 aa, Artificial Sequence
  PolpTN2?90-96?205-211?243-254?311-923
MSSLRPSSIIIDIYKVIQDHPDSSRWAIELRFLPKPISSEWIFITDIEERASEIDKVLTKYNIMKKKDAYVSMAIHDFEKVTAKLKRVQETEGERRLRRITLDKIQGDSETTSGFTLALVVDIDNTKIHDTRIIENEEEAFEASKREWEALKPKLQELGFLPRWILYTGGGLQLWFVSDKLEPISVIDRASEIIPNIMNGLLSEGFKADNIFDPARIVRAPLTFNHKYRTPTQVKGRVIEFNDVRISLTEFLDRLEAYAKEKGIPLEKPTKRKFLELASKRYEVTS

SEQ ID NO: 14 - protein, 928 aa, Thermococcus sp. CIR10
  Thermococcus sp. CIR10 DNA primase
MSGREFKRPSDVIIDIYKVIQDHPEAGRLAIEFRFYPYPTSEWILLNDIEDKAREIDKVLFKNNILGKKEAYISMAIHDFDEVTKKLEKLQELEHEKAQKEGRQPKEITLRHVQGEATGKIHTTVSSYTLTLVVDIDVNEIHDSKAVESEEKALEVSKRAWEVLKPNLEELGIKPRYVFFTGGGIQLWFVAPEPENISVIDKAAEIIPPVLNTLLPEGYSVDNIFDRARIVRVPFTVNYKYKTPDGKPLELRGRLLEFNDVRTPLGDILEKLEAYAKGHKISLGSTSRSGKFRGVAGRYEVKKENFEELAKRLVEELAPWFKKIKERGGSRHHLVNAIAAYIARNTNLTEEDLLGKDQEDGTHVVGLWELVHSKLVELGLEDPGDWSNRYHTIKDVYEKLYAGTTLGTRAYMMKYLNVSEEEAIEILRSVKRALFPYLHPVNVQVISKFEAKPYSKEEAPTEWEAVDEDRKKAVGIWYIEVLALETANYVYIEDLSKPGVFYIVEKVKRTVKVGKKEKGVEVDEYHFNPALWQSFLNWLGIKEGEPIEREELWNLLLEKFDIKDYELRAIYFRKILHLLSPEGMRRPRCVEEFLRELADEGFLSEDKVRHLAHWIKFYAKPLRHSTTSIMLRAKGKPVDMRMAVWAKVVEFFAEDEETAQGLIETFREAYEQAEPPFPCFGARECPFFQEHRGCPFIAPKRDEILAVSLVDVQLHGSDGIVIIVGSEEGTKKFVHKGKVEWQKQGKSKIKYPVAEWFLDVYAKEFLSLPEAPSWSHEEVTEILKSRARVVRSQLNEFDEYFDNFIDWLRSENARGIYPYEKADSSHIFIKGNMIGIPPRLAEDFYRNELGISGRKFKEMLIRELGSYYLGKKAAWIKLSSGQHNGVNCYFISLDWFKKIVGEPNIKDIEAEGDIGSGGFNYEEEEGEA

SEQ ID NO: 15 - protein, 303 aa, Artificial Sequence
  PolpCIR10?303-928
MSGREFKRPSDVIIDIYKVIQDHPEAGRLAIEFRFYPYPTSEWILLNDIEDKAREIDKVLFKNNILGKKEAYISMAIHDFDEVTKKLEKLQELEHEKAQKEGRQPKEITLRHVQGEATGKIHTTVSSYTLTLVVDIDVNEIHDSKAVESEEKALEVSKRAWEVLKPNLEELGIKPRYVFFTGGGIQLWFVAPEPENISVIDKAAEIIPPVLNTLLPEGYSVDNIFDRARIVRVPFTVNYKYKTPDGKPLELRGRLLEFNDVRTPLGDILEKLEAYAKGHKISLGSTSRSGKFRGVAGRYEVKK

SEQ ID NO: 16 - protein, 297 aa, Artificial Sequence
  PolpCIR10?93-98?303-928
MSGREFKRPSDVIIDIYKVIQDHPEAGRLAIEFRFYPYPTSEWILLNDIEDKAREIDKVLFKNNILGKKEAYISMAIHDFDEVTKKLEKLQEQKEGRQPKEITLRHVQGEATGKIHTTVSSYTLTLVVDIDVNEIHDSKAVESEEKALEVSKRAWEVLKPNLEELGIKPRYVFFTGGGIQLWFVAPEPENISVIDKAAEIIPPVLNTLLPEGYSVDNIFDRARIVRVPFTVNYKYKTPDGKPLELRGRLLEFNDVRTPLGDILEKLEAYAKGHKISLGSTSRSGKFRGVAGRYEVKK

SEQ ID NO: 17 - protein, 914 aa, Thermococcus peptonophilus
  Thermococcus peptonophilus DNA primase
MSELTPGKVLADVYKVIQDHPEAGRLAIELRFYPVIKSEWVLLNDIEDKARDIDKVLAKQNLIKGKEAYVSMAIHGFEAVKKKLEKLRESVEEGKVRKLGLENVQGEAKGKVHPTVSNYTLTLVVDVDIEAVHKLKVVEDVDKVFEKAKEGWLALKPVFEELGVLPRYVFFTGGGLQLWFVAPKLEDIAVIDRASGIVPNVLNALLPEGFVVDNIFDRARIVRAPLTVNHKYKAPNGARVGVKGRLIEFNDVRVSLSEVLDKLEVYAKERGIQLGGQEKVRGGRGFVNVRYVVKKEELETLALNLADELIPWFKKVKERGGSWHHLVNAIGAYVVRNTNLSLEDLIGKDNPDGTHVVGLWEIVFQRLVEKSAEDPGDWVNRRNTIKDVYEKHIAGKPLGTRAYLKKYLPVSDEEVVEILMAVRRALLPFLKEVKKVDSSGFGVAPYKKTAPRSWDEVDEDRKRATGRWYVWKLAFNTAEYLFTDELPKAGTFYIDVWMKEGKREWMKRFFNEALFRSFIEDGLGYKYGAPVEREELFERLVEVFNITDEEVRGIYIDAALSLLSPVGMRTPPCIEEFIMEFAANGSLSEDKVRHLARWIKLYAKPLKHSTTTTKLVGAGYKVDMRMAVWAKLVEFFAEDDEVARELVRVFKEEYGAAEPPFTCIGTKTCQFYLNEKMCPFIIPKEKEILAVSLIDVQRHESDGLVVIVGGDKEVRTFVKKGNVEWVKKTERREKYPVAEWFIDVFATEYLSVSPDDLDVDLEEVTDILKSRARVVKSRLNELEDMYEKFVEWLKRENAVRGVLPYEKADFNHLFIKGNMIGIPPALAEEFYRFELNIKGSEFREMLEKKLGFHYTKKAVKLSVGEKKDVRRCYLVSLEWFRKVVGEPNVKDVVMAGDIALSGLVYYESGEEVVE

SEQ ID NO: 18 - protein, 295 aa, Artificial Sequence
  PolpTpep?295-914
MSELTPGKVLADVYKVIQDHPEAGRLAIELRFYPVIKSEWVLLNDIEDKARDIDKVLAKQNLIKGKEAYVSMAIHGFEAVKKKLEKLRESVEEGKVRKLGLENVQGEAKGKVHPTVSNYTLTLVVDVDIEAVHKLKVVEDVDKVFEKAKEGWLALKPVFEELGVLPRYVFFTGGGLQLWFVAPKLEDIAVIDRASGIVPNVLNALLPEGFVVDNIFDRARIVRAPLTVNHKYKAPNGARVGVKGRLIEFNDVRVSLSEVLDKLEVYAKERGIQLGGQEKVRGGRGFVNVRYVVKK

SEQ ID NO: 19 - protein, 913 aa, Thermococcus celericrescens
  Thermococcus celericrescens DNA primase
MSELTPGKVLADVYKVIQDHPEAGRLAIELRFYPVIKSEWVLLNDIEDKARDIDKVLAKKNIINGKEAYVSMAIHDFGAVKKKLEKLREKAEGERARRIGLENVQGEAKGKVHPTVSNYTLALVVDIDIEEVHKSRVVEDVEAVFERAKKGWLALRPVFEELGVLPRYVFFTGGGLQIWFVAPELEDIAVIDRASGIVPGVLNALLPEGFVVDNIFDRARIVRAPLTVNHKYKAPNGAGLGVKGRLIEFNDVRVSLSEVLDKLEAYAKEKGIQLGGQERASGVRVFGKVRYEVKKERLETLALNLADELAPWFKKVKERGGSWHHLVNAIGAYIVRNTNLSLEDLIGKDNPDGTHVVGLWELVFQRLVEKGAEDPSDWLNRRNTIKDVYEKHIAGKPLGTRAYLKKYLPVSDEEVVEILMAARRALLPFLKGVKRAGSSGFGVFPYKDTAPRSWNEVEAERKRATGLWYVWSLAFSTAEYIFTDELSKAGTFFILVKEGKSIKSVFNETLFRSFIEEGLGYKYGMPVRRDELFERLVEVFNITDEEVRGVYIDAALSLLSPVGMRTPPCIEEFIMEFAANGNLPEDKVRHLARWIKLYAKPLKHSTTTTKLVGAGYNVDMRMAVWAKLVEFFAEDDEVAGELVRIFKEEYKEAEPPFTCIGARTCPFYLKDDVAKMCPFIFPKEKEILAVSLIDVQRHESDGIVILVGGAREVRTFIKKGKVEWVKRTKKTEKYPIAEWFIDVFATEYLGVPPDNLDFDLKDVTGILKSRERVVPSQLNEVEDWYEKFVEWLKRENEVRGVLPYKKADVHHLFIKDNMIGIPPALAEEFYRYEANIKPSEFREMLEVKLSIHYKKKAVKLSVGEKKDVRRCYLVSLEWFRKVVGEPNVKDVVMAGDIALSGLVYYESGEEVVE

SEQ ID NO: 20 - protein, 295 aa, Artificial Sequence
  PolpTcele?295-913
MSELTPGKVLADVYKVIQDHPEAGRLAIELRFYPVIKSEWVLLNDIEDKARDIDKVLAKKNIINGKEAYVSMAIHDFGAVKKKLEKLREKAEGERARRIGLENVQGEAKGKVHPTVSNYTLALVVDIDIEEVHKSRVVEDVEAVFERAKKGWLALRPVFEELGVLPRYVFFTGGGLQIWFVAPELEDIAVIDRASGIVPGVLNALLPEGFVVDNIFDRARIVRAPLTVNHKYKAPNGAGLGVKGRLIEFNDVRVSLSEVLDKLEAYAKEKGIQLGGQERASGVRVFGKVRYEVKK

SEQ ID NO: 21 - protein, 296 aa, Artificial Sequence
  PolpP12?297-898
MRPSDIIIDVYKAIQDHPGAGKLAIELRFYPRPTSEWIIVADIEDKAEELHKVLFKNNVLGKKEAYISMALHDFEEVGKKLEKLRELEEERAQKEGRKPREVTLRNVQGEATGKVHKTVSKYTLTLVVDIDVEEIHKSKVVESEEKAFELAKRAWDELKPKLEGIGVKPRYVFFTGGGVQLWFVAPGLEPIEVIDRASRVIPPVLNAMLPEGYSVDNIFDRARIVRVPLTINYKYKTPDERPLEIRGRLIEFNDVRTPLGEVLDKLEAYAKEHGISLVTPSQARFIGTVGRYEVDK